(19) United States
(12) Patent Application Publication
(10) Pub. No.: US 2002/0148002 A1
(43) Pub. Date: Oct. 10, 2002

(54) Plasmids and methods for construction of non-redundant, saturation, gene-disruption plant libraries

(76) Inventor: Ray Wu, Ithaca, NY (US)

Correspondence Address: Michael L Goldman Esq, Nixon Peabody LLP, Clinton Square, PO Box 31051, Rochester, NY, 14603, US

(21) Appl. No.: 09/574,038

(22) Filed: May 18, 2000

Related U.S. Application Data
(60) Provisional application No. 60/134,830, filed on May 19, 1999.

(51) Int. Cl. C12N 15/82

(52) U.S. Cl. 800/278

(57) ABSTRACT

The present invention relates to a method of constructing a non-redundant, saturation, gene-disruption genomic library suitable for the functional analysis of the entire genome of the target plant. The invention also relates to unique plasmids for use in the method and plants transformed with such plasmids.

This application claims the benefit of U.S. Provisional Patent Application Serial No. 60/134,830, filed May 19, 1999.

### FIELD OF THE INVENTION

The present invention relates to the design and construction of a series of plasmids which are used to produce a non-redundant, saturation, gene-disruption plant library. A gene disruption library is considered to be similar to a mutant insertional library. This invention also relates to plants transformed with these plasmids, and the progeny of such plants.

### BACKGROUND OF THE INVENTION

An ultimate goal of many plant scientists is to identify and discover the function of each gene in plants. The use of molecular biology techniques allows for the manipulation of genomes directed to this objective. A plant genome project can be arbitrarily divided into three phases. Phase I involves mapping the genome by genetic and physical methods. Phase II involves cloning and sequencing all, or most, of the genes. Phase III involves determining the function of each gene, before or after the sequence of the entire genome or that of the cDNAs is known. For convenience, Phase III can be further divided into three steps. Step one is to construct an insertional-mutant library, with the goal of disrupting each gene separately. Step two is to determine the DNA sequence that flanks the inserted plasmid, and the chromosomal location of the inserted plasmid, in each mutant plant. Step three is to determine the function of each gene.

Rice is one of the most important food crops in the world because it is the major staple food for over two billion people. Rice production must be increased by 50% in the year 2030 to feed the projected growth of population. Understanding how rice genes function will help to increase rice yields. Rice is a convenient model system for studying gene function, because it has a relatively small genome and it was the earliest cereal plant to undergo transformation and regeneration procedures. Moreover, due to synteny of genes with other cereal plants, any information obtained on rice genes will likely be applicable to other important cereal crops, such as maize, wheat, and barley.

After about 10 years of efforts by many scientists, physical mapping of the rice genome was virtually completed several years ago. In April 2000, it was announced by the Monsanto Company that most of the rice genome sequences have been determined. Thus, the work in Phases I and II is essentially concluded. Small-scale Phase III work started several years ago, but progress has been slow, because the current methods of generating specific mutant lines are slow and imprecise.

A significant amount of genomic work has been carried out in Arabidopsis, because of the relatively small genome of Arabidopsis. Several partial gene-disruption libraries have already been made. One type of library uses T-DNA to disrupt the gene in the Arabidopsis genome, which includes some 8,000 T-DNA gene-disrupted “tagged” mutants (Feldmann et al., “A Dwarf Mutant of Arabidopsis Generated by T-DNA Insertion Mutagenesis,” Science 243:1351-1354 (1989)). A major disadvantage of T-DNA tagging, and similar approaches, is that one needs as many transformation events as the number of T-tagged mutants. Since transformation of Arabidopsis is efficient, it is now possible to obtain 100,000 T-DNA tagged mutants with brute force (Krysan et al., “T-DNA As an Insertional Mutagen in Arabidopsis,” Plant Cell 11: 2283-2290 (1999)). On the other hand, transformation of rice is much less efficient. It is not yet practical to obtain anywhere close to 200,000 T-DNA tagged rice mutants.

A second type of library makes use of an endogenous transposon, such as Mu in maize (Bensen et al., “Cloning and Characterization of the Maize An1 Gene,” Plant Cell 7: 75-84 (1995)); tos17 transposon in Rice (Hirochika et al., “Retrotransposons of Rice Involved in Mutations Induced by Tissue Culture,” Proc. Natl. Acad. Sci. USA 93:7783-7788 (1996)). Although a large number of insertional mutants can be obtained, a major disadvantage is that it is difficult to get desired revertants, especially if a large number of insertions are present in each plant.

A third type of library involves transferring mobile genomic sequences, known as transposable elements, or transposons, from one plant to other plants. Transposable elements are either autonomous or nonautonomous. Autonomous elements carry the gene(s) coding for the enzymes required for transposition, thus autonomous elements have the ability to excise and transpose. Nonautonomous elements do not transpose spontaneously. They become mobile only when an autonomous member of the same family is present elsewhere in the genome. One well-characterized plant transposon is the maize Activator (“Ac”) and Dissociation (“Ds”) family of transposable elements. The family is comprised of the autonomous element Ac, and the nonautonomous Ds element. Ds elements are not capable of autonomous transposition, but can be trans-activated to transpose by Ac (Hehl et al., “Induced Transposition of Ds by a Stable Ac in Crosses of Transgenic Tobacco Plants,” Mol. Gen. Genet. 217:53-59 (1989)). Thus, transposable elements, such as Ac/Ds of maize, can be transferred to other plants to generate a relatively small number if anchor plants (such as 500), and then to produce a much larger number of secondary insertional-mutant plant lines. The major advantage to this method is that one needs a relatively small number of anchor plant lines (such as several thousands) to generate a large population of secondary mutant plant lines (such as 200,000) after transposition (Hehl et al., “Induced Transposition of Ds by a Stable Ac in Crosses of Transgenic Tobacco Plants,” Mol. Gen. Genet. 217:53-59 (1989); Bancroft et al., “Transposition Pattern of the Maize Element Ds in Arabidopsis Thaliana,” Genetics 134:1211-1229 (1993)). Since over 70% of the insertional mutants in Arabidopsis have no readily visible phenotype, the Ac/Ds system was improved by using enhancer- and gene-trap plasmids (Sundaresan et al., “Patterns of Gene Action in Plant Development Revealed by Enhancer Trap and Gene Trap Transposable Elements,” Genes & Develop. 9:1797-1810 (1995)), which allow disrupted genes with no phenotype to be detected by expression of a reporter gene (such as Gus). So far, this type of library includes less than 15,000 Ac/Ds-tagged plant lines (Chin et al., “Molecular Analysis of Rice Plants Harboring An Ac/Ds Transposable Element-mediated Gene Trapping System,” Plant J. 19: 615-623 (1999)). Therefore, many additional plant lines are still needed to complete the library. Chin recently generated several hundred Ac/Ds-based insertional-mutant rice lines by using the gene- and enhancer-trap approach (Chin et al., “Molecular Analysis of Rice Plants Harboring An Ac/Ds Transposable Element-Mediated Gene Trapping System,” Plant J. 19: 615-623 (1999)). Therefore, many more additional plant lines both in rice and Arabidopsis are still needed to produce a saturation library. One advantage of this type of insertional-mutant library is that it includes both gene tagging and knockout features. Another advantage of Ac/Ds-tagged plants is that revertants can be obtained relatively easily. However, the Ac/Ds tagged system also suffers from the same problem as T-DNA tagged plants, or use of an endogenous transposon to produce gene-disruption libraries because all of these libraries are constructed by a random “shotgun”-type approach. In any random approach, large amounts of time are wasted analyzing a high percentage of redundant plant lines. The general practice by most scientists is to generate and then analyze a tenfold larger excess of randomly generated plant lines to cover approximately 98% of the genome by calculation. For example, to achieve a 99% probability of tagging all the genes in the rice genome, 400,000 tagged plant lines are needed. The laboratory of Shimamoto obtained around 500 tagged mutant rice lines in 1993 (Shimamoto et al., “Trans-Activation and Stable Integration of the Maize Transposable Element Ds Cotransfected with the Ac Transposase Gene in Transgenic Rice Plants,” Mol. Gen. Genet. 239: 354-360 (1993)), and close to 8,000 last year (Enoki et al., “Ac as a Tool for the Functional Genomics of Rice,” The Plant J. 19:605-613 (1999)). There are at least three publications which show that after Ac/Ds-containing plasmids are integrated into the rice genome, transposition does occur and that the frequency of transposition in rice is relatively high, in the range of 3-15% (Shimamoto et al., “Trans-Activation and Stable Integration of the Maize Transposable Element Ds Cotransfected with the Ac Transposase Gene in Transgenic Rice Plants,” Mol. Gen. Genet. 239: 354-360 (1993); Enoki et al., “Ac as a Tool for the Functional Genomics of Rice,” The Plant J. 19:605-613 (1999); Chin et al., “Molecular Analysis of Rice Plants Harboring An Ac/Ds Transposable Element-mediated Gene Trapping System,” Plant J. 19: 615-623 (1999)).

Even though some methods are already available for studying the functions of individual genes in a genome, they are very time-consuming and labor intensive. It has been estimated that the amount of work needed for Phase III research (as described in the Background of the Invention Section) is on the order of ten times greater than the combined efforts of Phase I and II work. Within Phase III work, using the current methods, the time and effort needed for Steps two and three to analyze a saturation gene-disruption plant library are much more than those required for Step one. This is because in order to identify, for example, 25,000 independent and well-spaced gene-disrupted Arabidopsis plant lines, one may need to generate and then analyze 250,000 plant lines due to redundancy. The analysis includes determining the flanking DNA sequences, followed by looking for phenotypic, physiological, or biochemical changes in the 250,000 plant lines. Thus, improvements in the current methods are needed to make Phase III work faster and less labor-intensive. What is needed is a method which systematically tags all genes in a given plant genome, thereby eliminating the need for extreme redundancy in screening, and drastically reducing the time and labor required for gene identification. The present invention is directed to overcoming these and other deficiencies in the current art.

### SUMMARY OF THE INVENTION

The present invention relates to a method of constructing a non-redundant, saturation, gene-disruption plant library. This involves providing a plasmid having two clusters of unique enzyme-cutting sites and two dissociation elements, and transforming a plurality of plants with the plasmid to produce a plurality of transformed plants with the plasmid integrated at different locations within the genome of the plants. Next, the locations of the integrated plasmid in the transgenic plants are mapped to identify anchor transgenic plant lines with the integrated plasmid suitably spaced within the genome of the plants. Each of the homozygous anchor transgenic plant lines is then crossed with a plant having an activator element to form progeny plants. The crossing activates transposition of a portion of the plasmid bounded by the two dissociation elements to form a plurality of progeny plants having different genes disrupted. Next, the method of the present invention involves digesting the plant genome at different unique enzyme-cutting sites to release a DNA fragment from each of the transgenic progeny plants, and measuring the size of each of the released DNA fragments to determine transposition distances in each of the transgenic progeny plants. Next, progeny transgenic plants are selected with the transposition distances which are different than the transposition distances of the other progeny transgenic plants by a pre-determined amount to prepare a non-redundant, saturation, gene-disruption plant library.

The present invention also relates to a plasmid having an insert containing two dissociation elements and two clusters of unique enzyme-cutting sites. One cluster of unique enzyme-cutting sites is between the two dissociation elements in the insert, and the other cluster of unique enzyme-cutting sites is not between the two dissociation elements in the insert.

The present invention also relates to plants transformed with the plasmid of the present invention, and the progeny thereof.

By providing for an insertional-mutant library that is more complete, and less redundant than current methods, the present invention provides three major advantages. First, the present invention requires only a very small fraction of the time and labor currently needed to analyze the same number of plant lines. Second, the present invention requires sequencing only the flanking sequences by inverse PCR (or a faster method to be described herein) of those pre-selected plant lines without having to sequence a five- to tenfold redundant number of plants. Third, the method of the present invention leaves no gaps in this region or any other regions in the entire genome. In other words, all the genes can be systematically tagged (disrupted). Thus, the present invention provides an advantage over the published methods of constructing (Step one) as well as analyzing plant lines (in Steps two and three), by allowing for far more rapid analysis of the function of a very large number of genes in the genomes of any plant.

### BRIEF DESCRIPTION OF THE DRAWINGS

FIGS. 1A-D show the components of the super gene-trap plasmid, pSDsG. FIG. 1A shows details of the construction of plasmid pSDsG, designed for rice or monocot transformation. FIG. 1B is an abbreviated view of the components of pSDsG, shown without 3′ terminators. FIG. 1C shows the abbreviated structure of the pSDsG plasmid following integration and transposition. FIG. 1D is a plasmid similar to pSDsG with two lysozyme MAR sequences added.

FIG. 2 shows an abbreviated view of the structure and components of an enhancer-trap plasmid, pSDsE, designed for transformation of rice or other monocot cells.

FIGS. 3A-B show the components of plasmids pSDsG and pSDsE, for dicot transformation. FIG. 3A shows an abbreviated super gene-trap plasmid, pSDsG, for dicot transformation. FIG. 3B shows an abbreviated super enhancer-trap plasmid, pSDsE, for dicot transformation, which has the Arabidopsis Act2 minimal promoter (AAMP) included.

FIGS. 4A-C show an abbreviated view of three Ac-containing plasmids. FIG. 4A is an Ac-containing plasmid for transforming monocots, such as rice. It also contains a tobacco matrix attachment region (TMAR) sequence. FIG. 4B shows an Ac-containing plasmid with an inducible promoter (DMIP). FIG. 4C shows an Ac-containing plasmid for transforming dicots such as Arabidopsis, which includes two tobacco MAR sequences.

FIGS. 5A-B are schematic diagrams of the main steps of the method of the present invention, detailed as Stages I-VII. The steps following Ac-containing transformation occurs along the “A” line, and steps following Ds-containing transformation occurs along the “B” line.

FIG. 6 shows a PCR amplification scheme for use in determining the physical location of inserted plasmids in transformed Ds-containing plants.

FIGS. 7A-B show an analysis of transgenic plants for determining the location (distance) of transposition. FIG. 7A shows Anchor line A before transposition. FIG. 7B shows F2 plant lines #1-#10 after transposition of the Ds-containing segment.

FIG. 8 is an abbreviated physical map of the components around the original integration site in anchor Plant A.

FIGS. 9A-B illustrate the analysis of an F2 plant line in which the Ds-containing segment from pSDsG is assumed to be transposed to a location approximately 80 kb away from the anchor position. FIG. 9A is an expanded map of the right-hand side of Anchor line A in FIG. 7A, before transposition. FIG. 9B shows the same Anchor line after transposition.

FIG. 10 shows the determination of the transposition distance in subline #9 from FIG. 7B.

FIG. 11 shows an expanded map of the right-hand side of Anchor line A before transposition, where ER1, ER2, ER3, etc., are the approximate location of EcoRI sites on the right-hand side of A.

FIG. 12 shows the location of the transposed plasmid in plant line A-2. The position of the reinserted Ds-containing part of the plasmid is shown in the center of this figure, which includes the marker Gus gene.

FIG. 13 shows transformed plant A-4 after transposition, where the distance of transposition is approximately 37 kb from Ipo2 site in A (the distance may be 37 kb±3 kb), and an SR2 site is known to be approximately 33 kb from the Ipo2 site.

FIG. 14 shows the components of a Ds-containing plasmid, pEDI, which includes two I-PpoI sites, for transformation of Arabidopsis.

### DETAILED DESCRIPTION OF THE INVENTION

The present invention relates to a method of constructing a non-redundant, saturation, gene-disruption plant library. This involves providing a plasmid having two clusters of unique enzyme-cutting sites and two dissociation elements, and transforming a plurality of plants with the plasmid to produce a plurality of transformed plants with the plasmid integrated at different locations within the genome of the plants. Next, the locations of the integrated plasmid in the transgenic plants are mapped to identify anchor transgenic plant lines with the integrated plasmid suitably spaced within the genome of the plants. Each of the homozygous anchor transgenic plant lines is then crossed with a plant having an activator element to form progeny plants. The crossing activates transposition of a portion of the plasmid bounded by the two dissociation elements to form a plurality of progeny plants having different genes disrupted. Next, the method of the present invention involves digesting the plant genome at different unique enzyme-cutting sites to release a DNA fragment from each of the transgenic progeny plants, and measuring the size of each of the released DNA fragments to determine transposition distances in each of the transgenic progeny plants. Next, progeny transgenic plants are selected with the transposition distances which are different than the transposition distances of the other progeny transgenic plants by a pre-determined amount to prepare a non-redundant, saturation, gene-disruption plant library.

The present invention also relates to a plasmid having an insert containing two dissociation elements and two clusters of unique enzyme-cutting sites. One cluster of unique enzyme-cutting sites is between the two dissociation elements in the insert, and the other cluster of unique enzyme-cutting sites is not between the two dissociation elements in the insert.

The present invention also relates to plants transformed with the plasmid of the present invention, and the progeny thereof.

In accordance with the method of the present invention, two exemplary Ds-containing “super plasmids” were constructed. Each plasmid contains two maize Ds elements and two clusters of relatively rare enzyme-cutting sites, which allows the construction of non-redundant, saturation, gene-disruption plant libraries. While different components can be combined to create a plasmid with the ability to transform various types of plants (monocots and dicots) and animals, the plasmid of the invention is generally constructed as follows. Table 1 provides a list of abbreviations for the components of the plasmids to be described herein.

TABLE 1

| Abbreviation | Represents |
|---|---|
| 3 or 3SA | Triple splice acceptor sequence from |
| | a rice gene |
| 35P | CaMV 35S promoter |
| 35T | CaMV 35S 3′ terminator sequence |
| Ac | Activation sequence of maize |
| A4P | Rice Actin 4 promoter |
| AAI | Arabidopsis Act2 intron |
| AAP | Arabidopsis Act2 promoter, or a |
| | similar strong promoter for dicot |
| | plants |
| AI | Rice Actin 1 intron (Act1 intron) |
| AAMP | Arabidopsis Act2 minimal promoter |
| AP or Act Pro | Rice Actin 1 promoter or a similar |
| | strong promoter from a cereal plant |
| RAMP or Act100 P | Rice Actin-100 minimal promoter |
| Bar | Phosphinothricin acetyl transferase |
| | gene to confer herbicide resistance |
| Ds | Dissociation sequence of maize |
| DMIP | Dexamethasone inducible promoter |
| GapP or Gapc Pro | Arabidopsis cytoplasmic |
| | glyceraldehyde 3-P dehydrogenase |
| | promoter |
| GFP | Green Fluorescent Protein marker |
| | for selection |
| Gus | β-glucuronidase gene |
| Hyg | Hygromycin phosphotransferase |
| | gene for selection |
| I or Ipo | Synthetic oligonucleotide sequence |
| | including the 15-bp recognition |
| | sequence of I-PpoI; where I-PpoI is |
| | an intron-encoded endonuclease |
| M | A partially deleted single-copy gene |
| | in the rice or Arabidopsis genome |
| | for rapid PCR-based copy number |
| | analysis; for rice, a 107-bp |
| | cytochrome c gene is used |
| MAR | Matrix attachment region |
| NosT | Nopoline synthase (Nos) 3′ |
| | terminator sequence |
| N or Not | NotI restriction enzyme recognition |
| | sequence; when more than one |
| | identical restriction enzyme |
| | recognition sequence, such as N, is |
| | present, they are designated as N1, |
| | N2, etc. |
| NPTII | Neomycin phosphotransferase II |
| | gene |
| Pin2 | Potato proteinase inhibitor II gene |
| PinP | Potato proteinase inhibitor II |
| | promoter |
| PinT | Potato proteinase inhibitor II 3′ |
| | terminator sequence |
| P or Pro | Promoter |
| S or Sma | SmaI recognition sequence |
| T | 3′ terminator sequence |
| TMAR | Tobacco matrix attachment region |
| | sequence |
| TPase | Maize Ac transposase gene |
| UP or UbiP | Maize ubiquitin promoter or a |
| | similar strong promoter from a |
| | cereal plant |
| V | Plasmid vector such as |
| | pCAMBIA1300, which includes the |
| | left border (LB) and right border |
| | (RB) sequence of T-DNA, or the |
| | plasmid pBluescript SK |

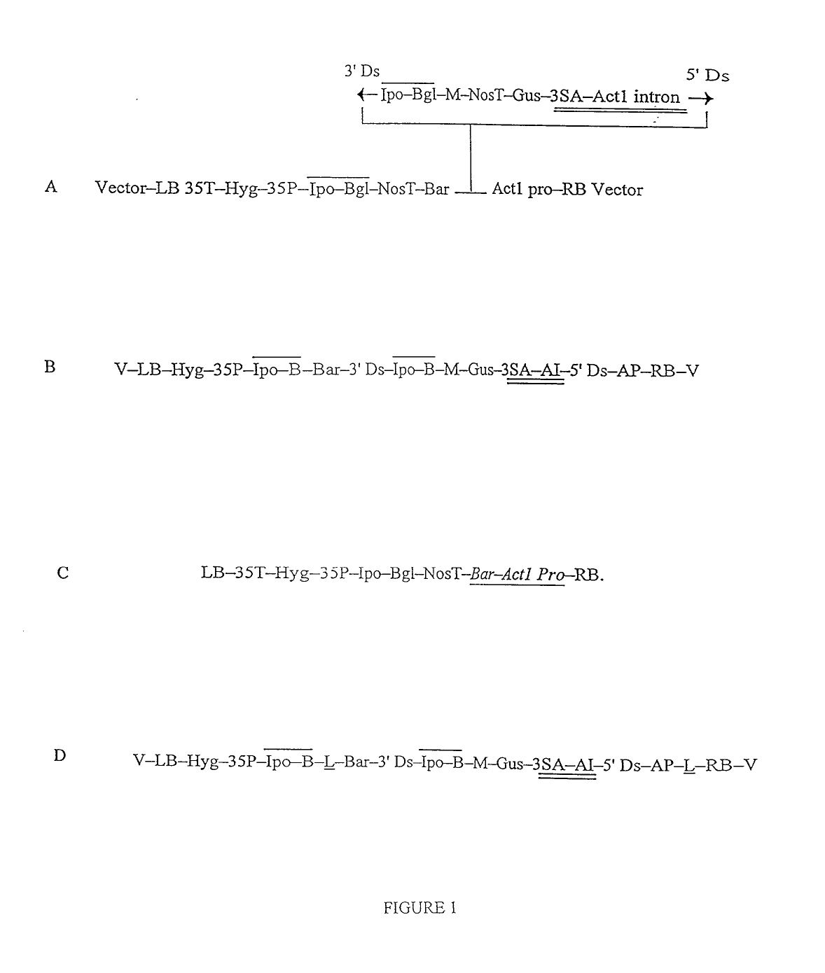

As a starting point for the plasmid of the present invention, an appropriate plant vector is chosen. For example, a plasmid vector such as pCAMBIA1300, which includes the left border (LB) and right border (RB) sequence of T-DNA or the phagemid pBluescript SK (Stratagene, La Jolla, Calif.) are suitable vectors. The plasmid is then constructed in such a way as to be useful for the species of the genome under study. The most important feature of this series of novel super plasmids is the inclusion of two identical clusters (or similar clusters) of enzyme recognition sequences placed in strategic locations in each super plasmid. This is because after transformation with a super plasmid to produce anchor plant lines, followed by Ac/Ds-mediated transposition in transgenic plants, the distance of transposition can be quickly and accurately measured (after enzyme digestion and gel electrophoresis) between the original anchor position to the newly transposed position in each plant line. These restriction sites include, but are not limited to I-PpoI, I-CeuI, AscI, NotI, PmeI, ApaI, BglI, and SmaI. The novel plasmids also include a gene-trap or enhancer-trap feature that includes a 13-glucuronidase gene (Gus), (Jefferson, “Assaying Chimeric Genes in Plants: The GUS Gene Fusion System,” Plant Mol Biol. Reporter 5:387-405 (1987), which is hereby incorporated by reference), or any other suitable reporter gene-containing cassette, which allows visualization of expression in the transgenic plants after transposition, even though there may not be readily detectable phenotypic changes in those plant lines. Thus, the gene trap and enhancer trap libraries are not only knockout libraries, but also have the additional feature of tagging and identifying plant lines and genes that have no visible phenotype. A partially deleted endogenous gene segment (designated as “M” herein) is also included in the plasmid, so that the transgene copy number in each plant, as well as the homozygosity of second or third generation plant lines, can be easily and rapidly determined by a PCR method. Finally, a selectable marker cassette, e.g., CaMV 35S promoter-Hyg (hygromycin phosphotransferase gene), is included for selection of transformed calli during transformation and regeneration of the plants. A second selectable marker cassette, e.g., Act1 promoter-Bar, is activated only after transposition in rice, such as is shown in FIG. 1.

In the gene trap system (also known as promoter trap and exon trap), the plasmid has no promoter. When a gene-trap plasmid disrupts a gene, it can detect the expression of a chromosomal gene (using the Gus reporter) when the Ds-containing segment is inserted within a transcribed region or the promoter region on the chromosome. Thus, the expression of Gus depends on the promoter in the rice chromosome. FIG. 1 shows the structure of a super gene-trap plasmid, pSDsG, for transformation of rice.

Promoters are chosen for inclusion in the construct in relation to the function of the particular plasmid. Promoters vary in their “strength” (i.e., their ability to promote transcription). For the purposes of expressing a cloned gene, it is usually desirable to use strong promoters in order to obtain a high level of transcription and, hence, expression of the gene. Suitable “strong” promoters for inclusion on the construct of the present invention include, but are not limited to, the maize ubiquitin promoter (Ubi) or a similar strong promoter from a cereal plant; the CaMV 35 S promoter; the glyceraldehyde 3-P dehydrogenase promoter of Arabidopsis (GapP), or an actin promoter, such as Act1Pro. In some instances, a weak, or “minimal” promoter is preferable, such as in the construct of the present invention known as a super enhancer gene, described in further detail herein. Examples of promoters appropriate for given applications are also further described below.

The DNA construct of the present invention also includes an operable 3′ regulatory region, selected from among those which are capable of providing correct transcription termination and polyadenylation of mRNA for expression in the host cell of choice, operably linked to the a DNA molecule which encodes for a protein of choice. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Nat'l Acad. Sci. USA 80:4803-4807 (1983), which is hereby incorporated by reference) and the cauliflower mosaic virus 3′ regulatory region (Odell, et al., “Identification of DNA Sequences Required for Activity of the Cauliflower Mosaic Virus 35S Promoter,” Nature 313(6005):810-812 (1985), which is hereby incorporated by reference). Virtually any 3′ regulatory region known to be operable in plants would suffice for proper expression of the coding sequence of the DNA construct of the present invention.

The vector of choice, enzyme recognition clusters, promoters, Ac or Ds elements, reporter cassettes, and an appropriate 3′ regulatory region can be ligated together to produce the plasmid of the present invention using well known molecular cloning techniques as described in Sambrook et al., Molecular Cloning: A Laboratory Manual, Second Edition, Cold Spring Harbor Press, NY (1989), which is hereby incorporated by reference.

FIGS. 1A and 1D show the structure of a super gene-trap plasmid, pSDsG, of the present invention for transformation of rice (Sundaresan et al., “Patterns of Gene Action in Plant Development Revealed by Enhancer Trap and Gene Trap Transposable Elements,” Genes & Develop. 9:1797-1810 (1995), which is hereby incorporated by reference). The gene-trap plasmid is designed to disrupt a gene and then detect the expression of a chromosomal gene (using the Gus or GFP reporter) when the Ds-containing segment is inserted within a transcribed region on the chromosome. The expression of Gus depends on the promoter in the chromosome. FIG. 1A shows a pSDsG for rice or monocot transformation. Note that the recognition sequences of two enzymes, Ipo-Bg1 (shown in FIG. 1A with a line on top of these sequences), represent only some of the recognition sequences. Many more recognition sequences are actually included in the plasmid, such as I-PpoI, I-CeuI, AscI, NotI, PmeI, ApaI, BglI, SmaI, SalI, XhoI, and EcoRI. The plasmid also includes a hygromycin phosphotransferase gene for selection purposes; a CaMV 35S promoter; a synthetic oligonucleotide sequence including the 15-bp recognition sequence of I-PpoI; where I-PpoI is an intron-encoded endonuclease (Muscarella et al., “Characterization of I-Ppo, an Intron-Encoded Endonuclease that Mediates Homing of a Group I Intron in the Ribosomal DNA of Physarum Polycephalum,” Mol. Cell Biol. 10:3386-3396 (1990), which is hereby incorporated by reference); a NotI restriction enzyme recognition sequence, shown as Not with a bar over it, (when more than one identical restriction enzyme recognition sequence, such as N, is present, they are designated as N1, N2, etc.); a Bar gene to confer herbicide resistance; two maize Ds sequences, a Gus gene for selection purposes, a rice Actin 1 intron and an rice Actin1 promoter, all which are operably fused into a plasmid vector. As can be seen, the Bar gene is now adjacent to the Act1 promoter, and thus activated and can synthesize phosphinothricin acetyl transferase to make the rice plant resistant to the herbicide Basta. Thus, this constitutes an easy and rapid way of recognizing a transposition event that is low in frequency (around 3-15%). This means that out of 100 F2 plants, transposition may have occurred in only 3 to 15 plants.

FIG. 1B is an abbreviated view of the components of pSDsG, shown without 3′ terminators. After integration of this plasmid in the rice genome and after transposition, the remaining part of the plasmid, including the empty site, has abbreviated structure shown in FIG. 1C. FIG. 1D shows a similar plasmid with two lysozyme matrix attachment regions (MAR) added.

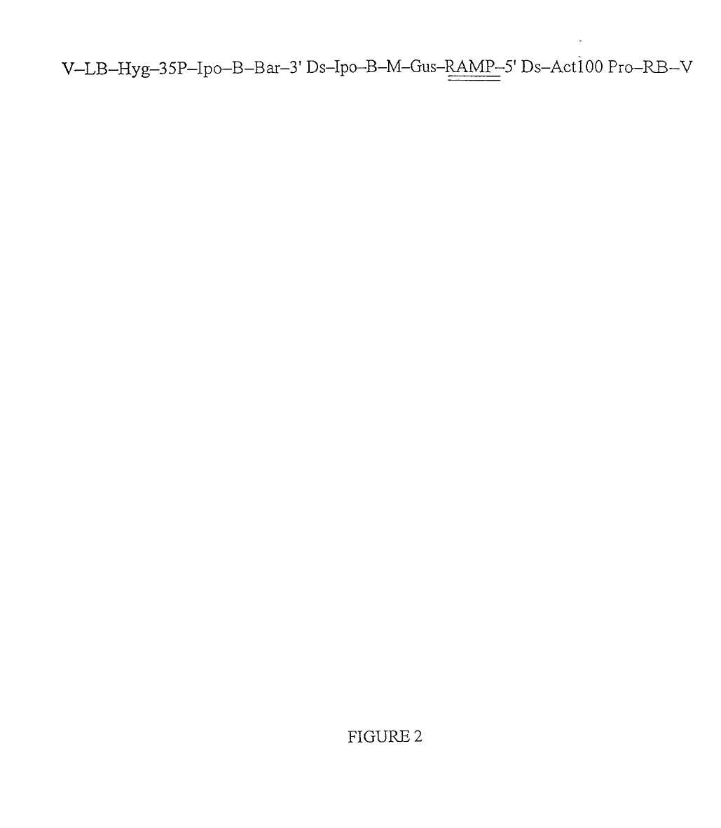

An example of a super enhancer-trap plasmid of the present invention, pSDsE, is shown in FIGS. 2A-B. In the enhancer-trap system, the plasmid has a minimal promoter that only expresses when inserted near a cis-acting enhancer in the chromosome. The pSDsE enhancer-trap plasmid includes a Gus gene, fused to a rice Act1-100 minimal promoter. The super enhancer-trap plasmid is designed so that expression of the Gus reporter gene is dependent on its insertion near chromosomal enhancer elements. Enhancer elements are DNA sequences located considerably up or downstream from the normal “startpoint” of a gene. Enhancer sequences resemble a promoter in terms of constitutive components, but enhancer elements are organized in a more closely packed array than promoter sequences. Enhancer regions contain elements that bind transcription factors, therefore, operationally, they resemble promoters. Most important is the fact that enhancer elements are not dependent on location for functionality. Enhancers can work bi-directionally, stimulating any promoter placed in the vicinity of the enhancer, even at a considerable distance from the gene's constitutive promoter. Thus, regardless of the orientation of the enhancer-trap plasmid following transposition (3′Ds→5′Ds or 5′Ds→3′Ds), or the distance from the transposition site to an endogenous promoter, the reporter gene is activated and the transposition site can be identified using the substrate 5-bromo-4-chloro-3-indolyl β-D-glucuronide (X-Gluc) according to the method described by Jefferson, “Assaying Chimeric Genes in Plants: The GUS Gene Fusion System,” Plant Mol Biol. Reporter 5:387-405 (1987), which is hereby incorporated by reference. The enhancer-trap plasmid of the present invention is designed to take advantage of the presence of endogenous enhancer elements in the target genome. The Gus gene of the enhancer-trap plasmid is fused to a minimal promoter derived from any suitable source. For example, a rice Act1-100 minimal promoter can be used for monocots, and a 47-bp minimal 35S promoter of CaMV can be used for dicots, as seen in FIG. 2A, with an abbreviated view of the components of the construct shown in FIG. 2B. Transposition of the Ds element to a site proximal to an enhancer region will “turn on” the promoter, allowing for identification of the transposition site, increasing the genes that can be identified as “tagged.” The super enhancer-trap plasmids share the same advantage of the super gene-trap plasmids in that the exact distance between the anchor site and the newly transposed site can be easily and accurately measured in a transgenic plant.

Using the gene-trap and enhancer trap super plasmids in concert increases the chances of tagging different genes in genome of a given transformed host cell, thereby reducing the number of transformed units to be analyzed.

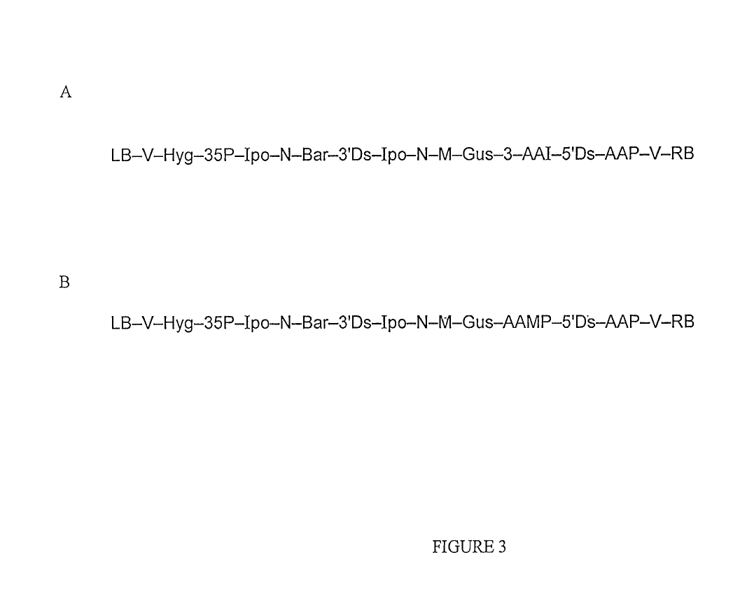

For transformation of dicots such as Arabidopsis, the 35S Cauliflower Mosaic virus promoter or cytoplasmic glyceraldehyde 3-P dehydrogenase promoter of Arabidopsis is used to replace the maize ubiquitin promoter; the Arabidopsis Act2 intron is used to replace the rice Act1 intron; and the Arabidopsis Act2 promoter is used to replace the rice Act1 promoter. In addition, the T-DNA left border (LB) and right border (RB) are always used to flank the plasmid, which are joined to the vector part of the plasmid as shown in FIG. 3. If the vector is pCAMBIA1300, the LB and RB are included automatically.

FIGS. 3A-B show the components of plasmids pSDsG and pSDsE, for dicot transformation. FIG. 3A is an abbreviated super gene-trap plasmid, pSDsG, useful for dicot transformation. In pSDsG, the Arabidopsis Act2 intron (AAI) and the Arabidopsis Act2 promoter (AAP) are included in the Ds element. FIG. 3B shows an abbreviated super enhancer-trap plasmid, pSDsE, for dicot transformation, where AAMP is the Arabidopsis Act2 minimal promoter.

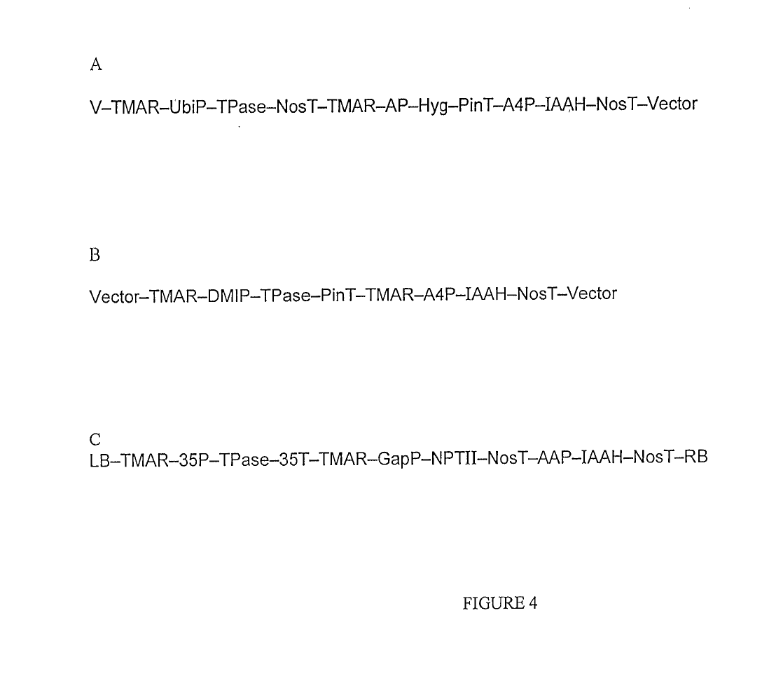

In addition to the Ds plasmids disclosed above, the present invention involves an Ac-containing plasmid. FIGS. 4A-C show representative Ac-containing plasmids. FIG. 4A is an Ac-containing plasmid for transforming monocots such as rice, where TMAR is a tobacco matrix attachment region sequence, and TPase is the maize Ac transposase gene and flanking sequences. The inclusion of the TMAR sequence increases the level of expression of the TPase gene and minimizes the chance of gene silencing (Spiker et al., “Nuclear Matrix Attachment Regions and Transgenic Expression in Plants,” Plant Physiol. 110:15-21 (1996); and Holmes-Davis et al., “Nuclear Matrix Attachment Regions and Plant Gene Expression,” Trends in Plant Science 3:91-96 (1998), which are hereby incorporated by reference). IAAH is an indole acetic acid hydrolase gene; it is used to eliminate plants that still harbor the Ac-containing plasmid after crossing an Ac-plant with a Ds-plant and allowing the progeny to segregate (Sundaresan et al., “Patterns of Gene Action in Plant Development Revealed by Enhancer Trap and Gene Trap Transposable Elements,” Genes & Develop. 9:1797-1810 (1995), which is hereby incorporated by reference).

FIG. 4B is an Ac-containing plasmid with an inducible promoter for co-transformation of monocots, where DMIP is the dexamethasone inducible promoter (Aoyama et al., “A Glucocorticoid-Mediated Transcriptional Induction System in Transgenic Plants,” Plant J. 11: 605-612 (1997), which is hereby incorporated by reference). When the plasmid shown in FIG. 4B is used for transformation, the Ac transposase gene is not expressed until the plants are sprayed with dexamethasone at suitable times. Other inducible promoters that may be used in place of DMIP include, but are not limited to, a jasmonate-inducible Pin2 promoter (Xu et al., “Systemic Induction of a Potato pin2 Promoter by Wounding, Methyl Jasmonate and Abscisic Acid in Transgenic Rice Plants,” Plant Mol. Biol. 22:573-588 (1993); Ryan, “Protease Inhibitors in Plants: Genes for Improving Defense Against Insects and Pathogens,” Annu. Rev. Phytopath. 28:25-49 (1990), which are hereby incorporated by reference), a heat-shock inducible promoter (Balcells et al., “A Heat-Shock Promoter Fusion to the Ac Transposase Gene Drives Inducible Transposition of a Ds Element During Arabidopsis Embryo Development,” Plant J. 5: 755-764 (1994), which is hereby incorporated by reference), and a low-temperature inducible (COR) promoter (Gilmour et al., “cDNA Sequence Analysis and Expression of Two Cold-Regulated Genes of Arabidopsis thaliana,” Plant Mol. Biol. 18:13-21 (1992), which is hereby incorporated by reference).

Two Ac-containing plasmids which are suitable for transforming dicots such as Arabidopsis in the present invention include the Ac-containing plasmid published by Sundaresan et al., “Patterns of Gene Action in Plant Development Revealed by Enhancer Trap and Gene Trap Transposable Elements,” Genes & Develop. 9:1797-1810 (1995), which is hereby incorporated by reference, and the plasmid shown in FIG. 4C, which includes two tobacco MAR sequences to increase the level of gene expression.

Instead of using the maize Ac/Ds system to produce a gene-disruption library, other transposable elements, such as Mu (for a review, see Walbot, “Strategies for Mutagenesis and Gene Cloning Using Transposon Tagging and T-DNA Insertional Mutagenesis,” Annu. Rev. Plant Physiol. Plant Mol. Biol. 43:49-82 (1992), which is hereby incorporated by reference), En/Spm (for a review, see Federoff, “Maize Transposable Elements,” Berg., eds., Mobile DNA, pp. 375-411 (1989), which is hereby incorporated by reference), etc., can be used.

A further aspect of the present invention includes a host cell which contains a DNA plasmid of the present invention. As described more fully hereinafter, the recombinant host cell can be either a bacterial cell (e.g., Agrobacterium) or a plant or animal cell. There are many methods of transformation into host cells known to those skilled in the art. The biolistic method (Cao et al., “Regeneration of Herbicide-Resistant Transgenic Rice Plants Following Microprojectile-Mediated Transformation Suspension Cells,” Plant Cell Reports 11:586-591 (1992),which is hereby incorporated by reference), which is also known as particle bombardment (U.S. Pat. Nos. 4,945,050, 5,036,006, and 5,100,792, all to Sanford, et al., which are hereby incorporated by reference), or the Agrobacterium-mediated method (Hiei et al., “Efficient Transformation of Rice (Oryza sativa L) Mediated by Agrobacterium and Sequence Analysis of the Boundaries of the T-DNA,” Plant J. 6:271-282 (1994), which is hereby incorporated by reference) are well suited for the transformation of rice, as well as many other plants. Recombinant constructs can also be introduced into cells via transduction, conjugation, mobilization, protoplast fusion, or electroporation (Fromm, et al., Proc. Natl. Acad. Sci. USA, 82:5824 (1985), which is hereby incorporated by reference). Other variations of transformation, now known to those skilled in art, or hereafter developed, can also be used. Suitable host cells include, but are not limited to, bacteria, virus, yeast, mammalian cells, insect, plant, and the like. Because the method of the present invention is particularly suited to reducing the time and labor spent reaching a functional understanding of the genome to which it is applied, many plants are suitable target cells for the method. These include, but are not limited to, cereal crop plants, such as barley, maize, and wheat; vegetables, such as soybeans, tomatoes, and broccoli; flowers, and fruit trees.

Following transformation, the cells are grown on a selective medium. Preferably, transformed cells are first identified using a selection marker simultaneously introduced into the host cells along with the DNA construct of the present invention. Suitable selection markers include, without limitation, markers coding for antibiotic resistance, such as the nptII gene which confers kanamycin resistance (Kan^{R})(Fraley, et al., Proc. Natl. Acad. Sci. USA, 80:4803-4807 (1983), which is hereby incorporated by reference); the IAAH gene, which confers resistance to naphthalene acetamide (“NAM”) (Sundaresan et al., “Patterns of Gene Action in Plant Development Revealed by Enhancer Trap and Gene Trap Transposable Elements,” Genes & Develop. 9:1797-1810 (1995), which is hereby incorporated by reference); the dhfr gene, which confers resistance to methotrexate (Bourouis et al., EMBO J. 2:1099-1104 (1983), which is hereby incorporated by reference); the Hyg gene, which confers resistance to hygromycin. Any known antibiotic-resistance marker can be used to transform and select transformed host cells in accordance with the present invention. Cells or tissues are grown on a selection media containing an antibiotic, whereby generally only those transformants expressing the antibiotic resistance marker continue to grow. Similarly, enzymes providing for production of a compound identifiable by color change are useful as selection markers, such as Gus (β-glucuronidase), or luminescence, such as luciferase.

Two approaches for transformation are involved in the present invention. In the first approach, plants are transformed either with a Ds-containing or an Ac-containing plasmid. After homozygous plants of each type are produced, a Ds-plasmid-containing plant is crossed with an Ac-plasmid-containing plant to produce F1 and F2 generation plants and to activate transposition of the Ds-containing plasmid in the plant chromosome. In the second approach, plants are co-transformed with two plasmids, one a Ds-containing plasmid and the other an Ac-containing plasmid. The transposase gene in this Ac-containing plasmid is linked to an inducible promoter. Thus, transposase gene expression can be activated only at the desired time to allow transposition of the Ds-containing plasmid in the same transgenic plant.

In the first approach, after transformation, the first step is to generate Ds-plasmid-containing anchor plant lines (primary gene-disrupted mutant plant lines); for example, approximately 150 lines are needed for Arabidopsis, and 500 for rice. The experimental design allows one to rapidly select one anchor plant line for approximately every 0.8-1.2 megabase pairs (Mb) of chromosomal DNA. After producing homozygous anchor plant lines, each line is crossed with an Ac-plasmid-containing plant to activate transposition of the Ds-containing plasmid in the F1 and F2 generation plants. In the second approach, after homozygous plants are produced, the inducible promoter is activated by the appropriate chemical/procedure to allow expression of the transposase gene, which then catalyzes transposition.

Next, the locations of the integrated plasmid in the transgenic plants are mapped to identify anchor transgenic plant lines with the integrated plasmid suitably spaced within the genome of the plants. Each of the homozygous anchor transgenic plant lines is then crossed with a plant having an activator element to form progeny plants. The crossing activates transposition of a portion of the plasmid bounded by the two dissociation elements to form a plurality of progeny plants having different genes disrupted. Next, the method of the present invention involves digesting the plant genome at different unique enzyme-cutting sites to release a DNA fragment from each of the transgenic progeny plants, and measuring the size of each of the released DNA fragments to determine transposition distances in each of the transgenic progeny plants. Next, the present invention involves selecting the progeny transgenic plants with the transposition distances which are different than the transposition distances of the other progeny transgenic plants by a pre-determined amount to prepare a non-redundant, saturation, gene-disruption plant library.

Preliminary Analysis of the Transgenic Plants

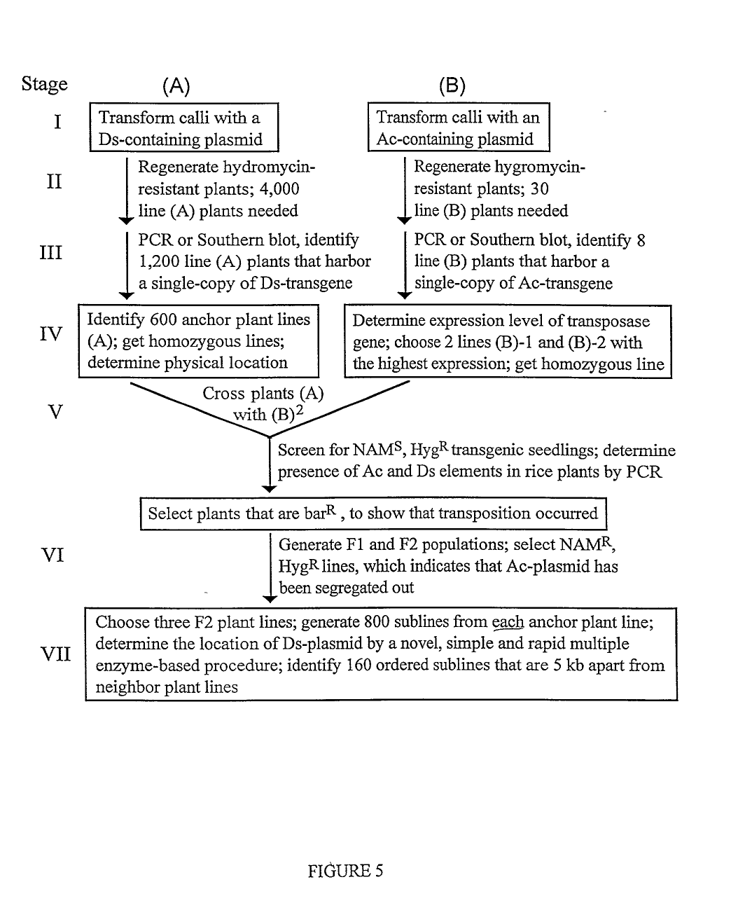

In this section, Cypress rice variety is used as an example to illustrate the principle and different analytic steps of the enzyme-based procedure of the present invention. Other plant varieties are appropriate for use with the method of the present invention, including the Nippon bare rice variety. FIG. 5 diagrams the steps, or Stages, I through VII, of the method of the present invention. These include simple procedures which are much faster than the different published procedures. Stages I and II, shown in FIG. 5, and described below, are essentially the same as those reported by Sundaresan et al., “Patterns of Gene Action in Plant Development Revealed by Enhancer Trap and Gene Trap Transposable Elements,” Genes & Develop. 9:1797-1810 (1995), which is hereby incorporated by reference. Stage III incorporates the simple and more rapid method of the present invention.

In Stage I, calli are transformed either with a Ds-containing plasmid, the “A” line of FIG. 5, or with an Ac-containing plasmid, shown in FIG. 5 as the “B” line. Next, as shown in FIG. 5, Stage II, “A” or “B” plants are grown in a medium containing a selectable marker. The transformed plants are identified by growth in hygromycin, and the hygromycin resistant plants which contain the Hyg gene are regenerated.

In Stage III, transgenic plants are chosen that contain only one or two copies of the transgene which harbor an unrearranged copy of the plasmid as shown in FIGS. 1A, 2A, and 3A. This is accomplished by either a standard polymerase chain reaction (PCR) (Erlich et al., “Recent Advances in the Polymerase Chain Reaction,” Science 252:1643-51 (1991), which is hereby incorporated by reference) or Southern blot analysis (Southern, “Detection of Specific Sequences Among DNA Fragments Separated by Gel Electrophoresis,” J. Mol. Biol., 98:503-17 (1975), which is hereby incorporated by reference), using primers complementary to the partially deleted single-copy endogenous gene segment that was included in the plasmid to detect the copy number of the deleted gene in comparison to the copy number of the normal gene in the allegedly transformed plant. An example of a suitable endogenous gene is a 1.7-bp cytochrome c gene. Homozygous R1 Ac-containing plants that harbor a single copy of the gene are used for further analysis.

At Stage IV, FIG. 5, the homozygous R1 Ac-containing plants, line “B,” are analyzed for the level of Ac expression. Since it is known that the Ac activity at different T-DNA insertion sites gives different levels of activity in Arabidopsis (Smith et al., “Characterization and Mapping of Ds-GUS-T-DNA Lines for Targeted Insertion,” Plant J. 10: 721-732 (1996) which is hereby incorporated by reference), it may be true in other plants, and a simple test can determine the level of Ac expression, thereby optimizing the system. The level of the transposase mRNA can be determined by RNA blot experiments. Two or three plants with the highest activity will be used to cross with Ds-containing plants.

Also in Stage IV, the approximate physical location of different anchor plant lines is determined for the Ds-containing transformants. Only those plant lines are chosen for further analysis that harbor a single copy of Ds-containing plasmids that are suitably distributed on the plant genome (e.g., approximately 800 kb apart from neighboring plant lines). If 600 anchor plant lines are identified, for example, the average distance will actually be 720 kb apart for rice, because the rice genome is 4.3×10⁵ kb. This is exemplary for the rice genome; for other plants the number of anchor lines needed will vary according to genome size. The physical determination is made by one of, or a combination of, the four following analytical procedures.

(1) The flanking sequence of each of the 1,600 plant lines, shown in Table 2, is determined, using the TAIL PCR method ((Liu et al., “Efficient Isolation and Mapping of Arabidopsis thaliana T-DNA Insert Junctions by Thermal Asymmetric Interlaced PCR,” Plant J. 8:457-463 (1995), which is hereby incorporated by reference), and the sequences are compared with the public databases. It is estimated that approximately 50% of the sequences will match those in the databases whose chromosomal locations are also known. Out of the 900 plant lines, it is likely that approximately 300 may be suitably spaced to become anchor plant lines.

Even though it is preferable to find 600 well distributed anchor plant lines, 400 is sufficient. If they are relatively equally distributed in the rice genome, the average distance between anchor plant lines will be 1,080 kb. Even if 300 anchor plant lines can be located, the average distance between anchor lines will be 1,430 kb (perhaps with a range between 1,000 kb and 1,900 kb). By adding more cycles of the chromosome-walking plan of the present invention, it is readily feasible to walk 1,000 kb from either side of an anchor plant line to cover a 2,000-kb (2 mb) region. If at least 300 well-spaced anchor plant lines can be obtained in this step, the remaining methods, (2) through (4) described below are not required. The 300 anchor plant lines can be used directly for Stage V, production of homozygous plant lines in R2.

(2) In the second method, chromosomal DNA is isolated from the leaves of transformed plants, digested with I-PpoI enzyme, followed by pulse-field gel electrophoresis (“PFGE”), and the size of the released DNA fragment is determined by probing with a telomere sequence (Liu et al., “Protection of Megabase-Sized Chromosomal DNA from Breakage by DNase Activity in Plant Nuclei,” BioTechniques 26: 258-26 (1999), which is hereby incorporated by reference). In this method, no flanking sequence needs to be determined. In principle, the physical location of the plasmid in anchor plant lines can be determined if the integrated copy of the I-PpoI-containing plasmid is within 10 mb from either end (telomeric region) of the chromosome. For example, in a 40-mb rice chromosome, in those plants in which the location of the integrated plasmid is within and up to 10 mb from each end, the location can be mapped by this method.

The error of this method for size determination is approximately ±8% of the distance between the inserted plasmid and one of the telomeres. For plant lines in which the physical location is within 3 mb from a telomere, the error is about ±0.2 mb with the current method, which is acceptable for the purpose of the present invention.

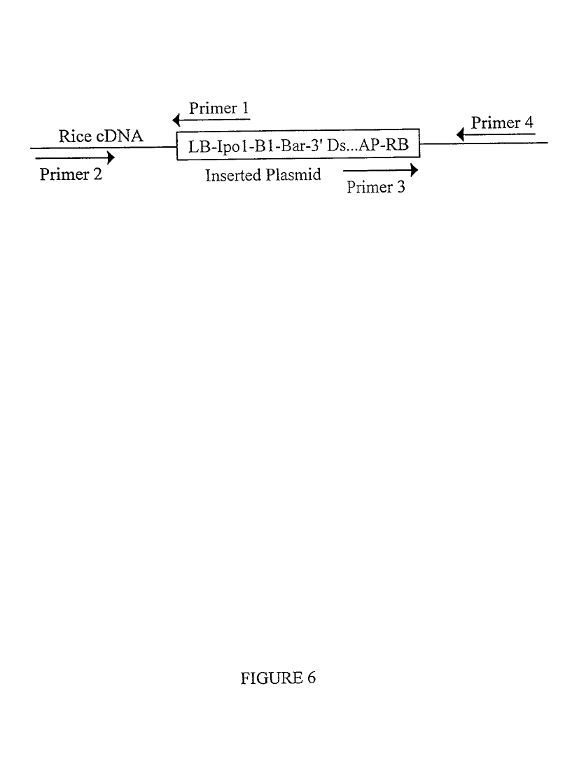

(3) In order to fill major gaps, if they exist, a PCR-based approach, as shown in FIG. 6, is used that does not require the determination of the flanking sequence of each inserted plasmid in different plant lines. This is accomplished by using a variation of the method reviewed by Walbot, “Strategies For Mutagenesis and Gene Cloning Using Transposon Tagging and T-DNA Insertional Mutagenesis,” Annu. Rev. Plant Physiol. Plant Mol. Biol. 43: 49-82 (1992), which is hereby incorporated by reference, and by Bensen et al., “Cloning and Characterization of the Maize An1 Gene,” Plant Cell 7: 75-84 (1995), which is hereby incorporated by reference, which involves the use of a pair of PCR primers, one from the end of the Ds-containing plasmid (for primer 1 and/or primer 3), and one from a known rice sequence (for primer 2 and/or primer 4), as shown in FIG. 6. A useful rice sequence includes a known gene, a cDNA, an RFLP or a SSLP marker (Bell et al., “Assignment of 30 Microsatellite Loci to the Linkage Map of Arabidopsis,” Genomics 19:137-144 (1994); Li et al., “Assignment of 44 Ds Insertions to the Linkage Map of Arabidopsis,” Plant Mol. Biol. Reporter 17:109-122 (1999), which are hereby incorporated by reference), that is already mapped on the rice chromosome with an accuracy of approximately 1 cM (230 kb), or is located on a mapped BAC clone. Any one from among several thousand sequences whose location is known can be utilized as a primer. Using rice as an example, approximately 2,000 sequences are chosen that are evenly distributed in the rice chromosomes (e.g., one sequence for approximately 800 kb or so to cover the entire rice genome). Primer sites for PCR amplification at this step are shown in FIG. 6. PCR amplification (e.g., between primer 2 and primer 1, or between primer 3 and primer 4, shown in FIG. 6) can occur only if the distance between a pair of primers is below 8 kb. A fragment of up to 8 kb can be produced by using a long-range DNA polymerase for PCR (Barnes et al., “PCR Amplification of Up to 35-kb DNA with High Fidelity and High Yield from Lambda Bacteriophage Templates,” Proc. Natl. Acad. Sci. USA 91:2216-2200 (1993), which is hereby incorporated by reference). Based on each of those sequences, primers 2 and 4 are synthesized and used for PCR. Any positive PCR result can be immediately used to define the physical location of the Ds-containing plasmid in an anchor plant line.

As soon as several anchor plant lines are located by any of the three methods, homozygous plant lines can be obtained from among the R2 generation. At the same time, some of the R2 plants during flowering stage will be crossed with an Ac-containing plasmid, as shown in Stage V, FIG. 5. After that, many F2 and F3 seeds will be collected from each cross to proceed with the analysis of sublines after transposition events have occurred.

(4) At this point, if there are gaps larger than 2 mb, it is possible that the gap regions may contain large stretches of repetitive sequences such as those around the centromere region. This can be checked with the DNA sequences in the public database. If this is the case, then this region will not need to be covered by making use of a larger number of sublines after transposition.

The next step in the method of the present invention involves obtaining homozygous anchor plant lines of second generation plants. This is shown as Stage V of FIG. 5. A homozygous Ac-plant is crossed with different homozygous Ds-plants, allowing transposition to occur, and many F1 generation plants are produced from 10 anchor plant lines. In some of these plants, transposition of the Ds element has occurred. Plants in which an inducible promoter is used are treated with the suitable inducing agent (e.g., dexamethasone for the glucocorticoid inducible promoter) at a time shortly before pollen mature or shortly after pollination. In this way, transposase is activated shortly after fertilization to allow germline transposition events to occur. Different F1 transgenic plants are allowed to self-pollinate and to produce many more F2 seeds. Among these plants, some seeds (approximately 25%) become homozygous by losing the Ac-containing plasmid (and the IAAH gene). Thus, the seedlings that germinate from these plant lines are NAM resistant (NAMR), and the NAMR, HygR transgenic rice seedlings are grown into plants. Next, a small amount of leaves from each plant is used to extract DNA to test, by PCR, whether transposition has occurred. PCR-positive plants are confirmed by Southern blot hybridization. The plants that show transposition give additional hybridizing bands when the SDsG fragment is used as the probe. Those plants that show transposition are selected by analyzed further by the method of the present invention, as described below, following generation of F1 and F2 populations, selected for, as shown in Stage VI of FIG. 5, for plants in which the Ac-plasmid has segregated out.

In case the anchor plant lines do not span the entire genome of a plant, Stage V of FIG. 5 can be repeated, starting with specific plant lines after the first transposition event to allow additional anchor plant lines to be produced.

Analysis of Plant Lines that Contain Transposed Ds-Associated Sequences to Determine the Distance of Different Transposition Events

The principle of the method for determining the distance of transposition between the anchor position and the position after transposition is discussed first.

Using current methods of analysis, the locations of the plasmid in the anchor position in a Ds plant, both before and after transposition, are determined by a genetic mapping method (Sundaresan et al., “Patterns of Gene Action in Plant Development Revealed by Enhancer Trap and Gene Trap Transposable Elements,” Genes & Develop. 9:1797-1810 (1995); Bancroft et al., “Transposition Pattern of the Maize Element Ds in Arabidopsis Thaliana,” Genetics 134:1211-1229 (1993), which are hereby incorporated by reference). This genetic mapping method is very time-consuming, because it involves the RFLP method and requires a large recombinant inbred (RI) population. An additional problem is that genetic mapping does not give the precise physical location of the plasmids in different plant lines before and after transposition. Although the SSLP method, which is much faster than the RFLP method, can also be used for mapping Arabidopsis (Bell et al., “Assignment of 30 Microsatellite Loci to the Linkage Map of Arabidopsis,” Genomics 19:137-144 (1994); Li et al., “Assignment of 44 Ds Insertions to the Linkage Map of Arabidopsis,” Plant Mol. Biol. Reporter 17:109-122 (1999), which are hereby incorporated by reference), it also suffers from the same problem in not being able to give the precise physical location of the plasmids in different plant lines. The precision of either mapping method is likely to have an error of over 20 kb. Thus, investigators cannot choose those plant lines that have an integrated plasmid every 5 kb or so in the genome.

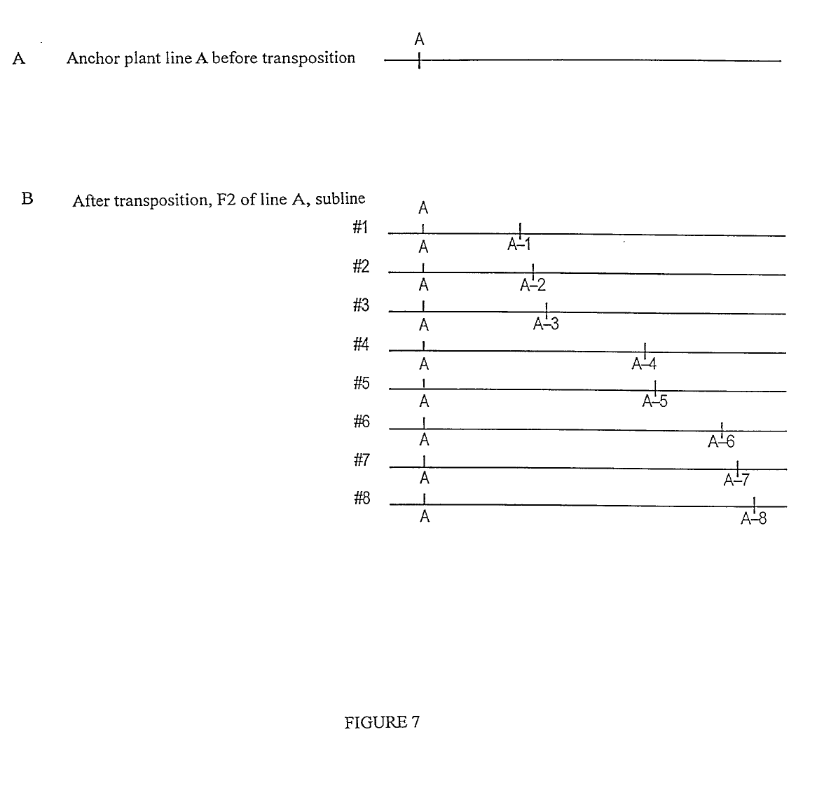

For the purpose of illustration, and to demonstrate how the published genetic-based methods are used, a 150-kb segment of a chromosome from the same anchor plant line A and 10 different F2 plant lines (sublines), instead of 120, are shown in positions 1 to 10 in FIG. 7B.

Analysis of Transgenic Plants Using the Published Genetic-Based Method.

FIG. 7 shows an analysis of transgenic plants for determining the location (distance) of transposition. The letter A in FIG. 7A represents the location of the integrated plasmid in anchor transgenic plant A. A-1, FIG. 7B, is the location of transposed and reintegrated Ds-containing portion of the integrated plasmid after transposition, a indicates the empty-site after transposition.

In this example, it is assumed that the exact distance of transposition is known, and the distance is written on top of each line in FIG. 7B. For example, in plant #1, location Al may be approximately 50 kb away from location a etc. Thus, out of these 10 plant lines, since the locations of the newly transposed sequences in sublines #1, #2, and #3 are very close, so are sublines #4, #5, and #6, they are redundant in tagging the same gene. Therefore, only one out of 3 lines are useful in tagging a gene of interest.

As can be seen from FIG. 7B, several large and small gaps exist in the 150-kb DNA fragment, because only 10 sublines are placed instead of 120 in this figure. The major difficulty is the genetic method cannot tell how many of these 10 sublines in FIG. 7B (120 sublines are actually generated) are redundant in tagging the same gene, thus most of the 120 sublines need to be analyzed by time-consuming procedures from this step on, including steps 2 and 3 of Phase III analysis. A comparison between our proposed systematic approach of producing insertional-mutant rice libraries and those already published is shown below in Table 2.

TABLE 2
Comparison of Five Methods to Construct a Saturation Gene-Disruption
Rice Library for Functional Genomics¹

| | | | Number of | | |
|---|---|---|---|---|---|
| | | | mutant | Can one | |
| | Method of | Number of | plant lines | identify | |
| | constructing | primary | need to be | mutants | Ease of |
| | an insertional- | transformants | extensively | with no | obtaining |
| Method | mutant library | needed² | analyzed⁴ | phenotype? | revertants |
| A | T-DNA | 1,200,000 | 400,000 | No | Difficult |
| | method^{a} | | (400,000)⁵ | | |
| B | Tos17 system^{b} | 12,000³ | 400,000 | No | Difficult |
| | | | (400,000)⁵ | | |
| C | Ac/Ds system^{c} | 12,000 | 400,000 | No | Easy |
| | | (3,600) | (400,000)⁵ | | |
| D | Ac/Ds system | 12,000 | 400,000 | Yes | Easy |
| | plus gene and | (3,600) | (400,000)⁵ | | |
| | enhancer | | | | |
| | traps^{d} | | | | |
| E | Method of the | 5,000 (1,600) | 96,000 | Yes | Easy |
| | present | | (3,000) | | |
| | invention | | or less⁶ | | |
| | (similar | | | | |
| | to D, but much | | | | |
| | Improved) | | | | |
| ¹Note that all of the numbers in this table have been estimated, based on known facts and assumptions. The numbers may vary ±30% without affecting the general principle of our approach. To achieve a 99% probability that every rice gene (5 kb apart) has been tagged, the well-known formula is used from any statistics textbook: P = 1 − (1 − f)ⁿ or n = ln(1 − P)/ln(1 − f) | | | | | |
| # (see Krysan et al., “T-DNA As an Insertional Mutagen in Arabidopsis,” Plant Cell 11: 2283-2290 (1999), for the source of formula and simple calculation), where P is the probability and f is the average distance (density) of genes in rice. n is the number of insertional mutants needed. For rice, P = 1 − (1 − [5/430,000])ⁿ, and thus n = 400,000. | | | | | |
| ^{a}Feldmann, K. A., “T-DNA Insertion Mutagenesis in Arabidopsis: Mutational Spectrum,” Plant J. 1: 71-83 (1991). ^{b}Hirochika, H., “Retrotransposons of Rice as a Tool for Forward and Reverse Genetics,” In Molecular Biology of Rice (Shimamoto, K., ed.), Springer, pp. 43-58 (1999); assume that each plant has 5 copies of the endogenous Tos17 transposon. ^{c}Shimamoto et al., “Trans-Activation and Stable Integration of the Maize Transposable | | | | | |
| # Element Ds Cotransfected with the Ac Transposase Gene in Transgenic Rice Plants,” Mol. Gen. Genet. 239: 354-360 (1993). ^{d}Sundaresan et al. “Patterns of Gene Action in Plant Development Revealed by Enhancer Trap and Gene Trap Transposable Elements,” Genes & Develop. 9: 1797-1810 (1995). | | | | | |
| ²According to the published results from the laboratory of Komari (Hiei et al., “Efficient Transformation of Rice (Oryza sativa L) Mediated by Agrobacterium and Sequence Analysis of the Boundaries of the T-DNA,” Plant J. 6: 271-282 (1994), which is hereby incorporated by reference)) using the Agrobacterium-mediated method for transformation, and our own data, approximately 30% of the transformants have a single copy of the transgene. Thus, to | | | | | |
| # compensate for this observation, one needs to obtain 3 fold more initial transformants if one wishes to work with only those plants that have a single copy of the transgene. Here 5,000 primary transformants will be produced, out of which approximately 1,600 are likely to harbor only one copy of the integrated plasmid in order to select 600 well-spaced anchor plant lines. Thus, numbers in parentheses are the expected number of rice plants with a single copy of the transgene. | | | | | |
| ³Assuming the tissue culture procedure to activate Tos17 transposon (Hirochika, H., “Retrotransposons of Rice as a Tool for Forward and Reverse Genetics,” In Molecular Biology of Rice (Shimamoto, K., ed.), Springer, pp. 43-58 (1999), which is hereby incorporated by reference, is equivalent to transformation of rice cells by the Ac/Ds system. | | | | | |
| ⁴Number of sublines of rice plants that need to be analyzed to achieve a 99% probability that every gene has been tagged. | | | | | |
| ⁵Numbers in parentheses indicate the number of flanking sequences that need to be determined. Assuming that only one (not both) flanking sequence for each insertional mutant line is sufficient. Many fewer flanking sequences need to be determined by the method of the present invention, because our pre-selected final sublines are linked to specific anchor plant lines. On the contrary, all other mutant libraries produce sublines that are not linked, and thus | | | | | |
| # each one has to be analyzed separately. | | | | | |
| ⁶96,000 final, ordered plant lines resulted after the rapid pre-selection of approximately 400,000 random sublines. To determine the location of the 600 anchor plant lines, the flanking sequences do not need to be sequenced. To determine the location of all sublines, the maximum number of flanking sequences that need to be determined is estimated to be 3,000 at the most. However, if the flanking sequence of an anchor line and a long stretch of sequences on | | | | | |
| # both sides is known and match those in the databank, a much smaller number of flanking sequences than 3,000 needs to be determined. | | | | | |

In analyzing the insertional mutant plant lines in the field to look for altered phenotypes, assuming that 5 plants of each mutant line needs to be planted, with any of the shotgun method generated mutant plant lines, 2,000,000 plants need to be planted and examined for phenotype changes. In contrast, with systematically generated mutant plant lines, one needs only to plant and examine 480,000 plants, which is only 24% the number needed for randomly generated plants. In conclusion, as can be seen from Table 2, the method of the present invention (E) is much superior than all the published approaches (A-D).

Principal of Novel Biochemistry-Based Method

In contrast to the genetic-based method, the distance between plant lines or sublines can be can rapidly and accurately measured by the method of the present invention. The method disclosed herein has three major advantages. First, only a small fraction of the time and labor is needed to analyze the same number of plant lines for their chromosomal location. Second, for each pre-selected anchor plant line, it is necessary only to sequence the flanking sequences by TAIL PCR (Liu et al., “Thermal Asymmetric Interlaced PCR: Automatable Amplification and Sequencing of Insert and Fragments from P1 and YAC Clones for Chromosome Walking,” Genomics 10: 674-681 (1995), which is hereby incorporated by reference). Third, this method leaves practically no gaps in this 150-kb region or any other regions in the entire genome. In other words, all the genes (chromosomal regions) can be systematically tagged.

Recall that for the construction of a saturation insertional-mutant rice library, only approximately 600 primary plant lines and 96,000 sublines need to be extensively analyzed. Moreover, the flanking sequences of less than 3,000 plant lines need to be determined because the different plant lines generated from the same anchor plant line are “linked.” This means that the approximate location of each subline is known relative to the location of the parent anchor line by the simple and rapid enzyme- and gel-based analysis of the present invention. If after determining the flanking sequence of a given anchor plant line, and perhaps several of the sublines within the 800-kb region, the sequence of that region, or certain segments within this region, is already known, then the work can be simplified. Thus, the method of the present invention has a tremendous benefit over the published shotgun methods of constructing (Step one) and analyzing the insertional-mutant plant lines (in Steps two and three).

In the design of the super plasmids of the present invention, each Ds-containing plasmid contains two clusters of enzyme recognition sequences (including I-PpoI, I-CeuI, SfiI, NotI, PmeI, ApaI and SmaI). Digestion of total plant chromosomal DNA is carried out by incubating with one of the enzymes that cleaves the DNA at two informative locations on the plant chromosomal DNA. One location is within the Ds elements, and the other is outside the Ds elements. For simplicity of illustration, only the relevant sites in anchor line A and F2 line A-1 to A-10 are shown in FIG. 7. Note that in anchor line A, before transposition, the components are based on those shown in FIG. 3A, but further abbreviated by including only relevant components.

Analysis of Transgenic Plants Resulting from a Single Anchor Plant Line, Using the Method of the Present Invention.

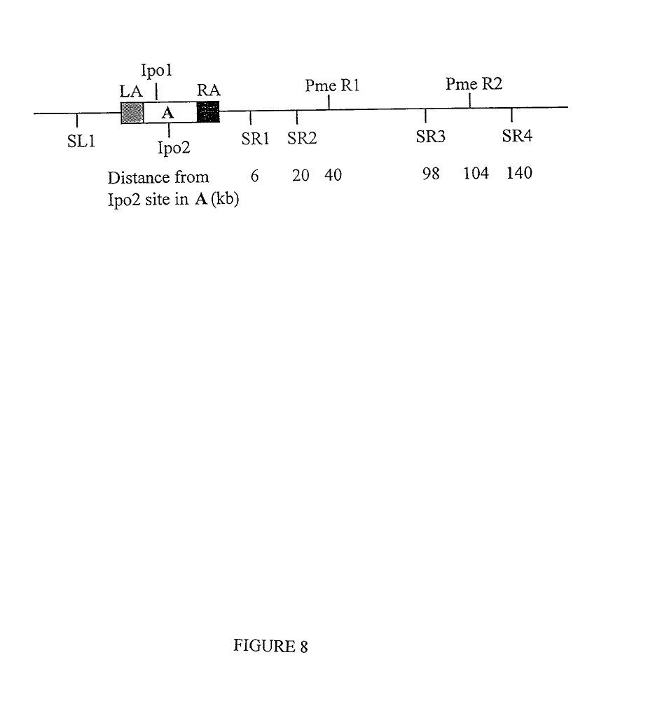

In Stage VI, shown in FIG. 5, F1 and F2 plant lines are chosen that have segregated out the Ac-containing plasmid, as indicated by the plant's resistance to NAM and Hyg. Next, in Stage VII, F2 plant lines are chosen for the next step in the analysis, which involves determining the location of the Ds-plasmid using the enzyme-based method of the present invention to determine the site of the plasmid insert before, and after translocation occurs. (1) First, the restriction sites surrounding the plasmid insertion site in anchor plant lines are determined. Information about the restriction sites surrounding the site of plasmid insertion into the anchor plant lines is needed to more accurately determine the transposition distance of many secondary plant lines that resulted after transposition. Selected restriction sites based on those present in the two clusters of enzyme-cutting sites in the plasmid are analyzed using Anchor plant line “A” as an example, shown in FIG. 8. FIG. 8 shows the restriction sites on the right-hand side of Anchor line A, in FIG. 7A, before transposition. SR1, SR2, SR3, etc. are the approximate location of SmaI sites on the right-hand side of plasmid A. LA represents the plant sequence immediately beyond the left border of the integrated plasmid, and RA represents the sequence beyond the right border of the plasmid. SR1 is a SmaI site on the right side of A. The steps for restriction site analysis are as follows:

(a) Determine flanking sequences on the left-side (LB) and right-side (RB) of plasmid insertion site in anchor plant A by using a traditional method, such as inverse PCR or TAIL PCR (Liu et al., “Efficient Isolation and Mapping of Arabidopsis thaliana T-DNA Insert Junctions by Thermal Asymmetric Interlaced PCR,” Plant J. 8:457-463 (1995), which is hereby incorporated by reference).

(b) Use LB and RB sequences separately as probes to determine the position of different restriction sites on both sides of integrated plasmid A as follows. First, digest genomic DNA with I-PpoI and SmaI, followed by agarose gel electrophoresis and hybridization. By using either the LA or RA sequence as the probe, the approximate distances between SL1 and A, as well as SR1 and A can be determined (based on the size of the hybridizing band). Similarly, digestion of genomic DNA with I-PpoI and PmeI shows the distances of Pme L1 and Pme R1 from the I-PpoI site (Ipo) in A. Finally, partial digestion with SmaI enzyme, and probing with RA, gives the approximate distances of SR2, SR3, etc. from Ipo site in integrated plasmid A.

Note that a partially digested plant DNA sample can be used also for many other probes, such as RB (right-hand flanking sequence of an anchor plant B), etc., to determine the restriction sites flanking other anchor plant lines (such as anchor plant B), etc.

(c) By using the same principle and other restriction enzymes, such as SfiI, NotI, etc., together with I-PpoI, to digest genomic DNA in anchor plant line A, one can reach at least 800 kb on the left-side and the right-side to span a region of approximately 1.6 megabase pairs (mb).

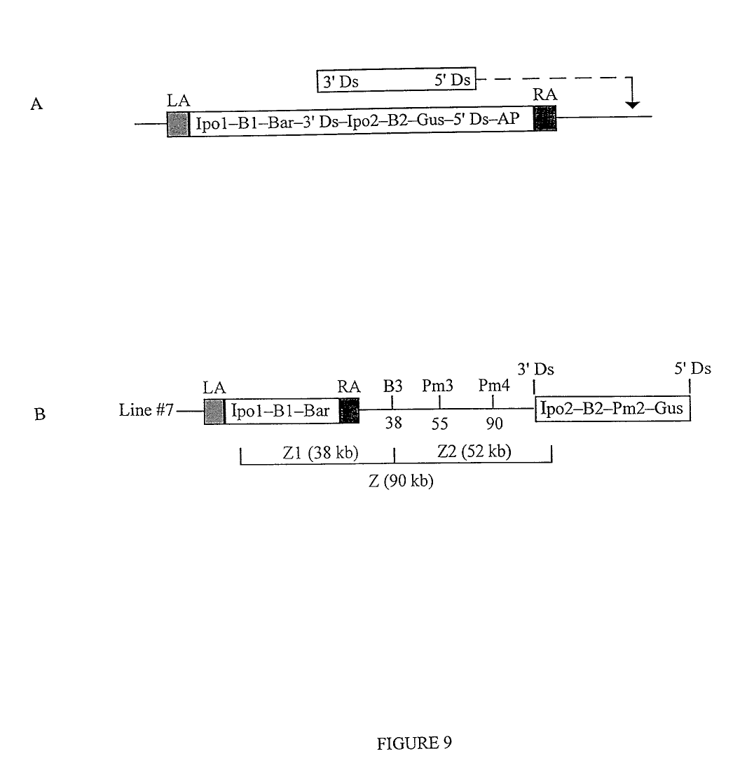

(2) Next, the plasmid transposition distances are determined. FIGS. 9A-B illustrate the analysis of an F2 plant line in which the Ds-containing segment from pSDsG is assumed to be transposed to a location approximately 80 kb away from the anchor position. Note in FIG. 9B that after transposition the Bar gene selectable marker is now adjacent to the AP promoter, and, thus, the plants become resistant to the herbicide phosphinothricin (or Basta). By using phosphinothricin for selection, those plant lines where transposition has occurred can be easily identified.

FIG. 9A shows Anchor line A before transposition (an abbreviated version of the plasmid is shown in FIG. 2). Abbreviations are the same as described above, except that LA represents the plant sequence immediately beyond the left border of the plasmid, and RA represents the plant sequence beyond the right border. Ipo1 and Ipo2 are the two Ipo sites; B1 and B2 are the two BglI sites. Open box(es) represent portions of the plasmid used for transformation; thin horizontal lines represent genomic DNA. After transposition, the DNA sequence within the borders of 3′ Ds and 5′ Ds will be transposed to a different location on the plant genome, as shown in FIG. 9B.

If the distance of transposition in different plant sublines is between 1 kb up to 50 kb, the transposition distance can be accurately determined by a commonly used simple procedure as follows. By digesting the chromosomal DNA with Ipo1, followed by agarose gel electrophoresis and probing with Bar, the size of the hybridizing band gives the distance of transposition. By this simple and rapid procedure, 1,000 plants can be analyzed within a few weeks. Out of these, it can be expected that a number of well-spaced sublines with transposition distances of approximately 5, 10, 15, 20, 25, 30, 35, 40, 45 and 50 kb from the anchor position will be found (such as A in FIG. 7B). It is also expected a number of plants will be found in which the transposition distance is between 50 and 100 kb. For example, it may not be possible to clearly distinguish the transposition distance of 80 kb from 85 kb. However, a more accurate determination of the distance can be made as follows.

As shown in FIG. 9B, if it is assumed the transposition distance is 90 kb, this distance can be measured more accurately by cleaving it into two smaller fragments and measuring the size of each. To achieve this goal, genomic DNA from plant line #7 (shown in FIG. 7B) is digested with I-PpoI enzyme to release a fragment Z. Following agarose gel electrophoresis (0.45% gel) and hybridization with 3′Ds DNA as the probe, the approximate size of fragment Z can be measured by comparison with several DNA size markers used during electrophoresis (the accuracy is approximately 90 kb +5 kb). The size of fragment Z is determined more accurately by digesting the genomic DNA with I-PpoI enzyme, plus another restriction enzyme such as BglI (B). Since on the average, the recognition sequence of BglI (B) is found every 20 kb in the plant genome (see Table 3, below), it is likely that fragment Z contains one or two BglI sites. If there is one BglI site such as B3 in fragment Z as in FIG. 9B, after digestion with BglI, the size of Z1 and Z2 can be determined accurately by using two different probes: one with the Bar sequence to detect fragment Z1, and the other with 3′Ds to detect fragment Z2. Since Z1 and Z2 are shorter than Z, the size of each fragment can be measured more accurately because electrophoretic mobility is a log function of molecular weight. In this example, Z1=38 kb, Z2=52 kb, and accuracy of measurement is ±2 kb. Similarly, the distance between Ipo1 and Pm3 can be determined (it is 55 kb in this example) after probing with Bar.

TABLE 3
Average Fragment Size of Restriction Enzyme-Digested Arabidopsis DNA*

| Enzyme | SfiI | AscI | NotI | PmeI | ApaI | BglI | SmaI | SalI | XhoI | EcoRI |
|---|---|---|---|---|---|---|---|---|---|---|
| Fragment | 400 | 400 | 200 | 60 | 25 | 20 | 10 | 6 | 4 | 4 |
| Size (kb) | | | | | | | | | | |

*(New England BioLabs Catalog 1998-99, p. 277)

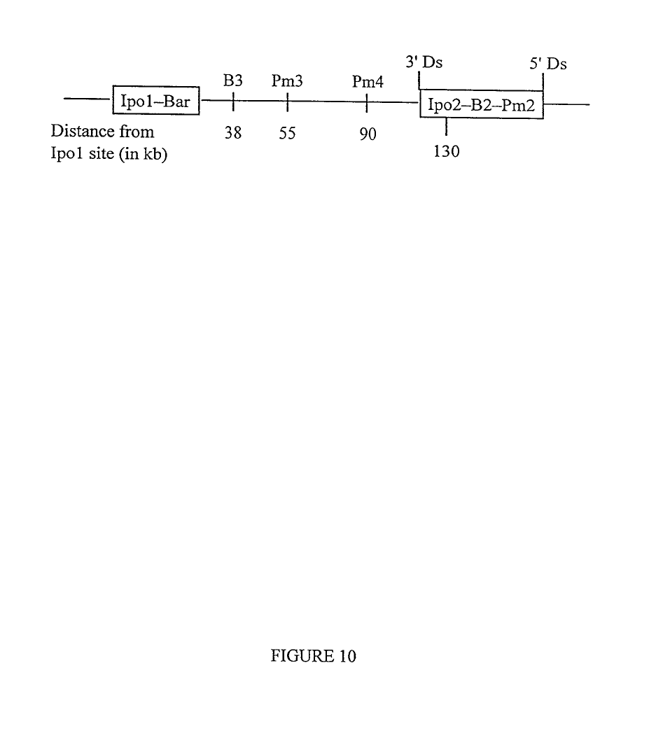

If the approximate distance of transposition in a particular subline is already determined, the distance can be measured more accurately by digesting genomic DNA with a specific enzyme and one of its recognition sequences, which is present within 50 kb from the left-hand of the 3′ Ds in this subline. This principle is illustrated by using the specific example shown in FIG. 10.

Relative to the original anchor position in plant A, assume that the approximate location of B3, Pm3, Pm4 has already been determined as shown in FIG. 9. First, the genomic DNA from subline #9 is digested with I-PpoI enzyme, followed by agarose gel electrophoresis and probing with Bar. In this example, it is assumed that the distance is approximately 130 kb±10 kb. The measurement can be made more accurate by digesting the genomic DNA with Pme1, followed by gel electrophoresis and probing with 3′ Ds. In this example, the fragment size between Pm4 and Ipo2 is 40 kb±0.2 kb. Since the distance between Ipo1 and Pm4 is already known to be 90 kb, then the distance of transposition in this subline #9 is 130 kb.

By repeating this process of specialized chromosome walking, step-by-step, the transposition distance of many other sublines can be determined relatively accurately and rapidly, because only ordinary agarose gel electrophoresis is needed. It is expected that this procedure can reach at least 400 kb to the right, and 400 kb to the left, from the original location of the Ds-containing plasmid in this anchor line A. Thus, a total distance of approximately 800 kb surrounding this or any other anchor line can be fully covered.

Analysis of many more F2 plant lines in which the Ds-containing segment from pSDsG is assumed to be transposed to many different locations, in different plant lines, all starting from a single anchor position, can be made in essentially the same manner by applying the method of the present invention.

Each anchor plant line (such as anchor line A) can be used to produce several thousands of F2 (or F3) sublines after transposition in order to span approximately 800 kb. Recall that the final aim of the present invention is to construct a saturation, insertional mutant library with an insertion in each 5 kb of the Arabidopsis and rice genome. Thus, approximately 160 F2 plant lines are needed to span the 800 kb adjacent to anchor line A. In order to obtain 160 suitably spaced F2 plant lines, approximately 800 F2 plant lines may need to be analyzed by agarose gel-based analysis. It is estimated that this can be accomplished by two scientists within a month.

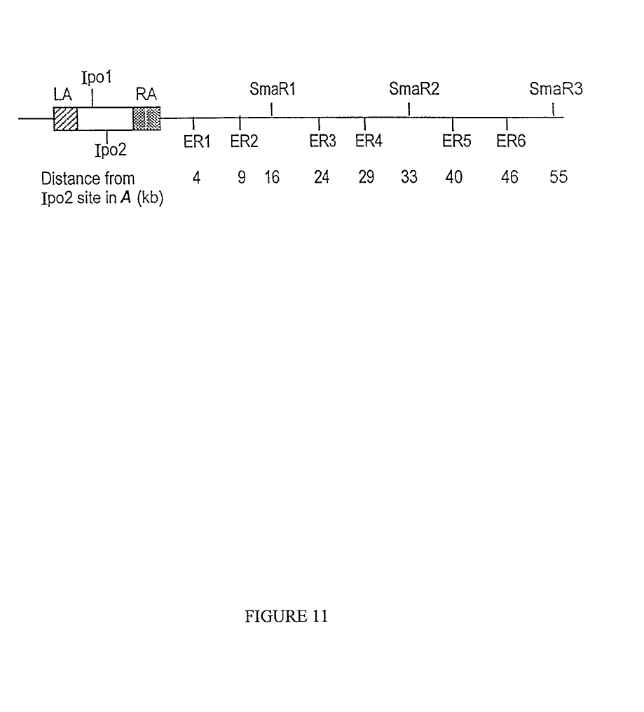

The determination of the transposition distance in different plant lines starting from anchor line A of FIG. 7A-B, using the method of the present invention for analysis, is demonstrated by FIG. 11. In this example, transposition distance is 50 kb. Estimation of the distance of transposition in each plant line, such as plant lines #1 to #10 in FIG. 7B, can be accurately determined as follows.

FIG. 11 shows an expanded map of the right-hand side of Anchor line A before transposition, where ER1, ER2, ER3, etc., are the approximate location of EcoRI sites on the right-hand side of A. This information is useful, because it helps one to decide which transgenic lines to analyze further by determining their flanking sequences. The flanking sequences of the inserted Ds-containing plasmid can be easily determined, and compared to those in the GenBank. If the sequence of this region of the genome is already known, then the location of ER1 to ER6 and SR1 to SR3 would also be known.

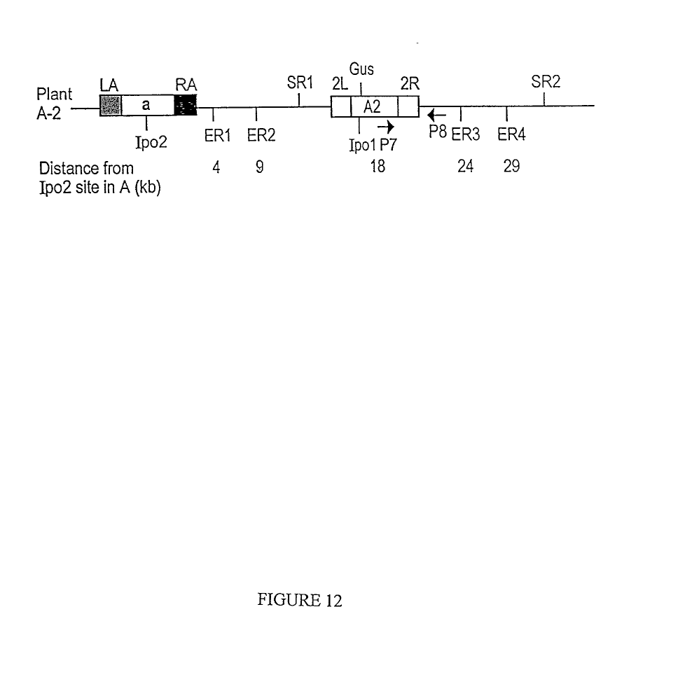

Another use of the plasmid of the present invention to determine sequences after transposition is shown in FIG. 12. FIG. 12 shows transformed plant A-2, where position of the reinserted Ds-containing part of the plasmid is shown as in the center of this figure, which includes the Gus marker, and where 2L and 2R represent the left- and right-side flanking sequences in plant A-2. After digesting the genomic DNA in plant A-2 with I-PpoI enzyme, followed by gel electrophoresis, the distance between the two Ipo sites can be determined accurately (in this example, 18 kb) by comparison with the mobility of DNA markers.

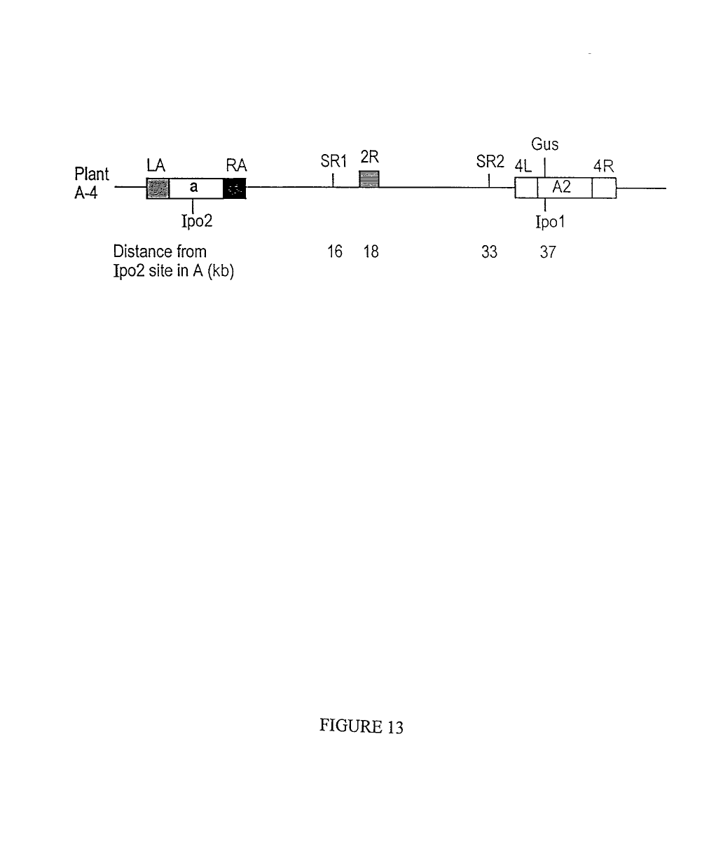

After discovering the approximate position of A2 in plant A-2, the flanking sequence on the right-hand side (2R) is determined by simple PCR as follows. If the sequence in this region is known by comparison with those in the GenBank, then by using primer 8 (P8, whose sequence is known) and primer 7 (P7, whose sequence is complementary to a portion of A2), the sequence between them can be amplified. Then by using primer 7 again, the sequence of the PCR product, including the 2R region, can be rapidly determined. If the sequence in this region, between ER3 site and Ipo1 site, is not known, then one can use the commonly adopted methods of inverse PCR or TAIL PCR (Liu et al., “Efficient Isolation and Mapping of Arabidopsis thaliana T-DNA Insert Junctions by Thermal Asymmetric Interlaced PCR,” Plant J. 8:457-463 (1995), which is hereby incorporated by reference). The sequence of 2R is then used as a probe to determine more exactly the distance of other plant lines such as plant A-4 as shown in FIG. 13.

In plant A-4, the distance of transposition is approximately 37 kb from Ipo2 site in A (the distance may be 37 kb +3 kb), and it is known that there is an SR2 site approximately 33 kb from the Ipo2 site, as seen in FIG. 13. In order to determine the distance between the Ipo2 and Ipo1 sites more accurately in plant A-4, the 2R probe in plant A-2 is used for hybridization (note that the position of 2R, which is the flanking sequence in the genome, is approximately 18 kb from Ipo2, but the DNA sequence between two Ds elements is not present next to 2R in plant A-4). The strategy is to measure the distance between the Ipo1 site and 2R, instead of between the Ipo1 and Ipo2 sites. In this example, genomic DNA in plant A-4 is digested with I-PpoI and SmaI enzyme (which cuts at SR1 and SR2), followed by gel electrophoresis. Then, by hybridizing with 2R as the probe, the hybridizing fragment size is determined to be 17 kb. Next, by using Gus as the probe, a fragment of 4 kb is found, which represents the distance between Hyg in A4 and the SR2 site. Since the distance between SR1 and Ipo2 is known to be 16 kb, then the distance between Ipo1 and Ipo2 is 16+17+4=37 kb. Here, the error of size estimation is reduced to approximately ±1 kb.

For determination of transposition distances of up to 600 kb, the type of analysis described with reference to FIG. 13 is repeated, resulting in accurate transposition distances in other transgenic lines. In the case of plant line A-4 shown in FIG. 13, the 4R flanking sequence of A-4 plant is determined and, then, 4R is used as the probe for the next set of plants. In principle, this type of selective chromosome walking can allow the accurate determination of the location of the transposed segment in many transgenic plant lines, up to at least 600 kb away from the anchor plasmid position. Similar analysis can be done using LB probe and place many plant lines in the left-hand side of the anchor plasmid in plant A.

The final result of the above analysis is that the accurate distance of transposition of many plant lines that are derived from the same anchor plant line A can be determined. By analyzing 600-800 plant lines, those plant lines can be chosen that have transposition distances approximately 5 kb between any adjacent plant lines. For example, it can expected that approximately 80 sublines (secondary plant lines) can be identified with transposition/reinsertion sites of approximately 5, 10, 15, and 20 kb, etc., up to 400 kb on the left-hand side, and 80 plant lines on the right-hand side of the integrated plasmid position in anchor plant A. In this method of analysis, it is not necessary to determine the flanking sequences of each of these 160 sublines, which span 800 kb of DNA. At the most, the determination of the flanking sequence of one plant line out of 10 plant lines is required. Thus, a large amount of time is saved by eliminating the need to carry out inverse PCR analysis on all 800 plant lines, which is required when the published shotgun procedures from other laboratories are utilized.

Since approach of the present invention is a systematic approach, assuming that 800 of the sublines are within a 800 kb region centered around an anchor line A, all these sublines are linked to the anchor line A, with approximate distance known after an enzyme-based analysis. Approximately 160 sublines will be selected out of this 800 kb region. The remaining 640 sublines are not useless, because they represent sublines that have insertions in this region with an average distance of 1 to 3 kb apart. Some of them may be useful in regions where the gene size is 2 or 3 kb instead of 5 kb. Thus, these sublines can be saved.

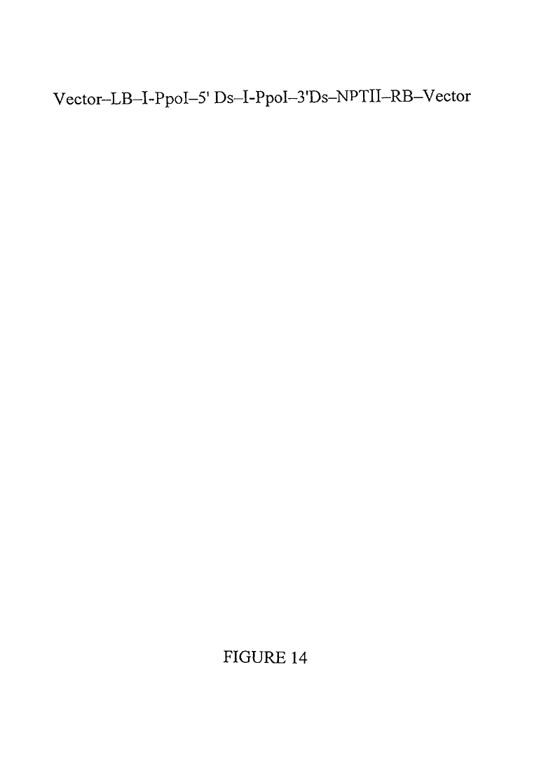

In order to test the validity of the principle of this invention, a simpler plasmid, pEDI, was first constructed. This plasmid, as shown in FIG. 14 in an abbreviated form, includes two I-PpoI sites, for transformation of Arabidopsis. Plasmid pEND4K (Klee et al., “Vectors For Transformation of Higher Plants,” Bio/Technology 3: 637-642 (1985), which is hereby incorporated by reference), is used as the vector. LB and RB are the left and right borders of the T-DNA, respectively. The 5′ Ds and 3′ DS sequence are from Hehl (Hehl et al., “Induced Transposition of Ds By a Stable Ac in Crosses of Transgenic Tobacco Plants,” Mol. Gen. Genet. 217: 53-59 (1989), which is hereby incorporated by reference). All other components of this plasmid are from commonly available sources. Methods for the construction of the pEDI used the common procedures as described in Ausubel et al., Current Protocols in Molecular Biology, Wiley, Supplement 29 (1993), which is hereby incorporated by reference. The plasmid is first tested by digestion with I-PpoI enzyme, and a 400-bp DNA fragment is released as expected.

Plasmid pEDI is transformed into A. thaliana C24 by an Agrobacterium-mediated method. First-generation plants are screened by germinating plants on agar plates that contain 30 mg/L of kanamycin. Kanamycin-resistant plants are obtained.

For illustration, Arabidopsis is used as an example to show the principle of the design and the method of the analysis of transgenic gene-disrupted plants in accordance with the present invention. The same principle can be used for any monocot or dicot, including the production of gene-disrupted mutants in trees. In principle, this invention can be applied to any plant species, as long as transformation and regeneration systems are available, and the Ac/Ds system can operate in that species (for reviews, see Federoff, “Maize Transposable Elements,” In: Mobile DNA (Berg, D. D. and Howe, M. M., eds.), pp. 375-411 (1989); Martienssen, “Functional Genomics: Probing Plant Gene Function and Expression with Transposons,” Proc. Natl. Acad. Sci. USA 95:2021-2026 (1998); Enoki et al., “Ac as a Tool for the Functional Genomics of Rice,” The Plant J. 19:605-613 (1999); Wu, “Report of the Committee on Genetic Engineering: Functional Genomics of Plants,” Rice Genetics Newsletter 16:10-14 (1999), which are hereby incorporated by reference).

### EXAMPLES

### Example 1

### Preliminary Analysis of Transgenic Arabidopsis Plants

Following transformation with pEDI as described above, over 700 first-generation plants were screened by germinating the seeds in the presence of kanamycin. Most plants were resistant to kanamycin, indicating that they harbor the pEDI plasmid. Second- and third-generation plants (R2 and R3) were screened again with kanamycin and the segregation pattern scored. Over 300 plants, which are shown to harbor a single copy of the pEDI plasmid, have become homozygous. R3 plants are further analyzed using molecular biology techniques.

### Example 2

### Analysis of Transgenic Arabidopsis Plants Using Molecular Biology Techniques

Out of 300 plant lines analyzed, over 50 are randomly selected for DNA blot hybridization (Southern blot) analysis. Each is shown to contain an integrated copy of the pEDI plasmid. Additional analysis is carried out on 39 transgenic plant lines by isolating the chromosomal DNA using the agarose embedding technique (Liu et al., “Thermal Asymmetric Interlaced PCR: Automatable Amplification and Sequencing of Insert and Fragments from P1 and YAC Clones for Chromosome Walking,” Genomics 10: 674-681 (1995), which is hereby incorporated by reference.) After preliminary pulsed-field gel electrophoresis (PFGE) for 8-12 hours to remove broken DNA, the DNA in the gel plug is removed and digested with I-PpoI enzymes. After longer PFGE (24-36 hours), the DNA in the gel is blotted onto nylon filters. DNA blot hybridization is carried out using the Arabidopsis telomere sequence as the probe. Hybridizing bands within the size range of 0.1 to 5 Mb are found in different samples, indicating that the fragments include a chromosomal end. Without further mapping the exact location of these plants, each plant (about 10) is used in the next step by crossing with Ac-containing plants.

### Example 3

### Crossing Ds-Containing Plants with Ac-Containing Plants

Each Ds-containing plant (that showed hybridizing bands after digesting the DNA with I-PpoI enzymes, followed by PFGE) is crossed with two different Ac-containing plants (lines Ac2 and Ac5), which are obtained from Sundaresan et al., “Patterns of Gene Action in Plant Development Revealed by Enhancer Trap and Gene Trap Transposable Elements,” Genes & Develop. 9:1797-1810 (1995), which is hereby incorporated by reference. Seeds from each cross are collected and germinated. A portion of each three-week-old F1 plantlet is used for PCR analysis to identify those plants in which transposition has occurred. Later on, PCR analyses are carried out with F2 plants. Those plants in which transposition has occurred give different patterns of PCR-produced DNA bands.

In the next step, DNA from the plants that show transposition is used for further analysis by digestion with the I-PpoI enzymes. Then, electrophoresis is carried out to look for the appearance of a new DNA band. Regular agarose gel electrophoresis is used first which can detect the appearance of new DNA bands with the size range of 2 kb to 50 kb. Those samples that give new DNA bands larger than 50 kb are further analyzed by PFGE. In both cases, the approximate size of the new DNA band gives the distance of transposition.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. A method of constructing a non-redundant, saturation, gene-disruption plant library comprising:
providing a plasmid having 2 clusters of unique enzyme-cutting sites and 2 dissociation elements;
transforming a plurality of plants with the plasmid to produce a plurality of transformed plants with the plasmid integrated at different locations within the genome of the plants;
mapping the locations of the integrated plasmid in the transgenic plants to identify anchor transgenic plant lines with the integrated plasmid suitably spaced within the genome of the plants;
crossing each of the homozygous anchor transgenic plant lines with a plant having an activator element to form progeny plants, wherein said crossing activates transposition of a portion of the plasmid bounded by the 2 dissociation elements to form a plurality of progeny plants having different genes disrupted;
digesting the plant genome at different unique enzyme-cutting sites to release a DNA fragment from each of the transgenic progeny plants;
measuring the size of each of the released DNA fragments to determine transposition distances in each of the transgenic progeny plants; and
selecting the progeny transgenic plants with the transposition distances which are different than the transposition distances of the other progeny transgenic plants by a pre-determined amount to prepare a non-redundant, saturation, gene-disruption plant library.

2. A method according to claim 1 further comprising:
sequencing regions flanking the integrated plasmid in selected progeny plants of the non-redundant, saturation, gene-disruption plant library to mark the disrupted genes.

3. A method according to claim 1 further comprising:
determining the function of the disrupted genes of the non-redundant, saturation, gene-disruption plant library.

4. A method according to claim 1, wherein said digesting is carried out by serial, separate use of a plurality of restriction enzymes specific to one of the unique enzyme cutting sites in the integrated plasmid.

5. A method according to claim 4, wherein said digesting is carried out by serial, separate use of different restriction enzymes, each specific to one of the unique enzyme-cutting sites, until the gene fragment is less than 30 kilobases.

6. A method according to claim 1, wherein the plasmid has an insert, wherein the insert comprises:
the 2 dissociation elements and
the 2 clusters of unique enzyme-cutting sites, wherein 1 cluster of unique enzyme-cutting sites is between the 2 dissociation elements in the insert and the other cluster of unique enzyme-cutting sites is not between the 2 dissociation elements in the insert.

7. A method according to claim 1, wherein the dissociation element is a maize dissociation element.

8. A method according to claim 1, wherein the cluster of unique enzyme-cutting sites is formed from 2 or more adjacent enzyme-cutting sites selected from the group consisting of I-PpoI, CeuI, AscI, NotI, PmeI, ApaI, BglI, SmaI, SalI, XhoI, and EcoRI.

9. A plasmid having an insert, wherein the insert comprises:
2 dissociation elements and
2 clusters of unique enzyme-cutting sites, wherein 1 cluster of unique enzyme-cutting sites is between the 2 dissociation elements in the insert and the other cluster of unique enzyme-cutting sites is not between the 2 dissociation elements in the insert.

10. A plasmid according to claim 9, wherein the dissociation element is a maize dissociation element.

11. A plasmid according to claim 9, wherein the cluster of unique enzyme-cutting sites is formed from 2 or more contiguous enzyme-cutting sites selected from the group consisting of I-PpoI, CeuI, AscI, NotI, PmeI, ApaI, BglI, SmaI, SalI, XhoI, and EcoRI.

12. A plant transformed with the plasmid according to claim 9.

13. A plant according to claim 12, wherein the dissociation element is a maize dissociation element.

14. A plant according to claim 12, wherein the cluster of unique enzyme-cutting sites is formed from 2 or more contiguous enzyme-cutting sites selected from the group consisting of I-PpoI, CeuI, AscI, NotI, PmeI, ApaI, BglI, SmaI, SalI, XhoI, and EcoRI.

15. A plant resulting from crossing a homozygous anchor plant derived from the plant according to claim 12 with a plant having an activator element.

16. A plant according to claim 15, wherein the dissociation element is a maize dissociation element.

17. A plant according to claim 15, wherein the cluster of unique enzyme-cutting sites is formed from 2 or more contiguous enzyme-cutting sites selected from the group consisting of I-PpoI, CeuI, AscI, NotI, PmeI, ApaI, BglI, SmaI, SalI, XhoI, and EcoRI.

18. A progeny plant produced from the plant according to claim 15.

19. A progeny plant according to claim 18, wherein the dissociation element is a maize dissociation element.

20. A progeny plant according to claim 18, wherein the cluster of unique enzyme-cutting sites is formed from 2 or more contiguous enzyme-cutting sites selected from the group consisting of I-PpoI, CeuI, AscI, NotI, PmeI, ApaI, BglI, SmaI, SalI, XhoI, and EcoRI.
